SEQUENCE LISTING (47 sequences)

SEQ ID NO: 1 - DNA, 20 bp, Artificial Sequence
  Synthetic
ggcttccacaccgtacagcg

SEQ ID NO: 2 - DNA, 20 bp, Artificial Sequence
  Synthetic
ctggctggaatcacggtagc

SEQ ID NO: 3 - DNA, 20 bp, Artificial Sequence
  Synthetic
gtgtgtgcgctgataatcac

SEQ ID NO: 4 - DNA, 20 bp, Artificial Sequence
  Synthetic
taaatcacagggccaacccg

SEQ ID NO: 5 - DNA, 20 bp, Artificial Sequence
  Synthetic
gtacatcccactgtaccaga

SEQ ID NO: 6 - DNA, 20 bp, Artificial Sequence
  Synthetic
tcgatgaagcccggcgcatc

SEQ ID NO: 7 - DNA, 20 bp, Artificial Sequence
  Synthetic
acaggtttcgttccgagaac

SEQ ID NO: 8 - DNA, 20 bp, Artificial Sequence
  Synthetic
gggcttcatcgacaccacgg

SEQ ID NO: 9 - DNA, 20 bp, Artificial Sequence
  Synthetic
gccattgacgtcatggcggc

SEQ ID NO: 10 - DNA, 20 bp, Artificial Sequence
  Synthetic
gagctaggggcttgtcatag

SEQ ID NO: 11 - DNA, 23 bp, Artificial Sequence
  Synthetic
ggcttccacaccgtacagcgtgg

SEQ ID NO: 12 - DNA, 23 bp, Artificial Sequence
  Synthetic
ctggctggaatcacggtagctgg

SEQ ID NO: 13 - DNA, 23 bp, Artificial Sequence
  Synthetic
gtgtgtgcgctgataatcacggg

SEQ ID NO: 14 - DNA, 23 bp, Artificial Sequence
  Synthetic
taaatcacagggccaacccgagg

SEQ ID NO: 15 - DNA, 23 bp, Artificial Sequence
  Synthetic
gtacatcccactgtaccagaggg

SEQ ID NO: 16 - DNA, 23 bp, Artificial Sequence
  Synthetic
tcgatgaagcccggcgcatccgg

SEQ ID NO: 17 - DNA, 23 bp, Artificial Sequence
  Synthetic
acaggtttcgttccgagaactgg

SEQ ID NO: 18 - DNA, 23 bp, Artificial Sequence
  Synthetic
gggcttcatcgacaccacggagg

SEQ ID NO: 19 - DNA, 23 bp, Artificial Sequence
  Synthetic
gccattgacgtcatggcggccgg

SEQ ID NO: 20 - DNA, 23 bp, Artificial Sequence
  Synthetic
gagctaggggcttgtcatagtgg

SEQ ID NO: 21 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 22 - protein, 1082 aa, Artificial Sequence
  Synthetic
MAAFKPNPINYILGLAIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIAAALPFSRTWDDSFNNKVLVLGSEAQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 23 - protein, 2414 aa, Artificial Sequence
  Synthetic
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 24 - protein, 617 aa, Artificial Sequence
  Synthetic
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 25 - protein, 3844 aa, Artificial Sequence
  Synthetic
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRAAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIHYPYDVPDYAS

SEQ ID NO: 26 - protein, 2048 aa, Artificial Sequence
  Synthetic
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDYPYDVPDYAS

SEQ ID NO: 27 - protein, 1726 aa, Artificial Sequence
  Synthetic
MAAFKPNPINYILGLAIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIAAALPFSRTWDDSFNNKVLVLGSEAQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRSRADPKKKRKVEASGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDYPYDVPDYAS

SEQ ID NO: 28 - protein, 22 aa, Artificial Sequence
  Synthetic
DYKDHDGDYKDHDIDYKDDDDK

SEQ ID NO: 29 - protein, 8 aa, Artificial Sequence
  Synthetic
PKKKRKVG

SEQ ID NO: 30 - protein, 10 aa, Artificial Sequence
  Synthetic
YPYDVPDYAS

SEQ ID NO: 31 - protein, 1368 aa, Artificial Sequence; identical to SEQ ID NO: 21
  Synthetic

SEQ ID NO: 32 - protein, 1082 aa, Artificial Sequence; identical to SEQ ID NO: 22
  Synthetic

SEQ ID NO: 33 - protein, 19 aa, Artificial Sequence
  Synthetic
ATNFSLLKQAGDVEENPGP

SEQ ID NO: 34 - protein, 1483 aa, Artificial Sequence
  Synthetic
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRADALDDFDLDMLGSDALDDFDLDMLGSDALDDFDLDMLGSDALDDFDLDMLINYPYDVPDYAS

SEQ ID NO: 35 - protein, 1155 aa, Artificial Sequence
  Synthetic
MAAFKPNPINYILGLAIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIAAALPFSRTWDDSFNNKVLVLGSEAQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRSRADPKKKRKVEASGSGRADALDDFDLDMLGSDALDDFDLDMLGSDALDDFDLDMLGSDALDDFDLDMLINSR

SEQ ID NO: 36 - protein, 295 aa, Artificial Sequence
  Synthetic
MDYKDHDGDYKDHDIDYKDDDDKMAPKKKRKVGRGMAQAALEPGEKPYACPECGKSFSDCRDLARHQRTHTGEKPYKCPECGKSFSRSDDLVRHQRTHTGEKPYKCPECGKSFSQSSNLVRHQRTHTGEKPYACPECGKSFSTSGELVRHQRTHTGEKPYKCPECGKSFSQRAHLERHQRTHTGEKPYKCPECGKSFSQAGHLASHQRTHTGKKTSGQAGQASPKKKRKVGRADALDDFDLDMLGSDALDDFDLDMLGSDALDDFDLDMLGSDALDDFDLDMLINYPYDVPDYAS

SEQ ID NO: 37 - protein, 860 aa, Artificial Sequence
  Synthetic
MDYKDHDGDYKDHDIDYKDDDDKMAPKKKRKVGRGMAQAALEPGEKPYACPECGKSFSDCRDLARHQRTHTGEKPYKCPECGKSFSRSDDLVRHQRTHTGEKPYKCPECGKSFSQSSNLVRHQRTHTGEKPYACPECGKSFSTSGELVRHQRTHTGEKPYKCPECGKSFSQRAHLERHQRTHTGEKPYKCPECGKSFSQAGHLASHQRTHTGKKTSGQAGQASPKKKRKVGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDYPYDVPDYAS

SEQ ID NO: 38 - DNA, 20 bp, Artificial Sequence
  Synthetic
ccccagacaggggtagtgcg

SEQ ID NO: 39 - DNA, 20 bp, Artificial Sequence
  Synthetic
gggtgacagtcgctggtcat

SEQ ID NO: 40 - DNA, 20 bp, Artificial Sequence
  Synthetic
ggcttctccgacaacttaaa

SEQ ID NO: 41 - DNA, 20 bp, Artificial Sequence
  Synthetic
tcataggatcctgaatcatt

SEQ ID NO: 42 - DNA, 20 bp, Artificial Sequence
  Synthetic
agccctccctgactcatgca

SEQ ID NO: 43 - DNA, 23 bp, Artificial Sequence
  Synthetic
ccccagacaggggtagtgcgagg

SEQ ID NO: 44 - DNA, 23 bp, Artificial Sequence
  Synthetic
gggtgacagtcgctggtcatagg

SEQ ID NO: 45 - DNA, 23 bp, Artificial Sequence
  Synthetic
ggcttctccgacaacttaaaagg

SEQ ID NO: 46 - DNA, 23 bp, Artificial Sequence
  Synthetic
tcataggatcctgaatcattagg

SEQ ID NO: 47 - DNA, 23 bp, Artificial Sequence
  Synthetic
agccctccctgactcatgcaggg

(19) United States
(12) Patent Application Publication
(10) Pub. No.: US 2019/0194633 A1
(43) Pub. Date: Jun. 27, 2019

(54) COMPOSITIONS, SYSTEMS AND METHODS FOR PROGRAMMING IMMUNE CELL FUNCTION THROUGH TARGETED GENE REGULATION

(71) Applicant: Duke University, Durham, NC (US)

(72) Inventors: Charles A. Gersbach, Durham, NC (US); Joseph J. Bellucci, Durham, NC (US)

(21) Appl. No.: 16/322,234

(22) PCT Filed: Aug. 10, 2017
(86) PCT No.: PCT/US2017/046282

§ 371 Date: Jan. 31, 2019

Related U.S. Application Data
(60) Provisional application No. 62/373,343, filed on Aug. 10, 2016.

(51) Int. Cl. C12N 9/22; C12N 15/11; C12N 15/10; C12N 5/0783; C12N 9/10

(52) U.S. Cl. CPC C12N 9/22; C12N 15/11; C12N 15/1093; C12N 5/0636; C12N 9/1029; C12Y 203/01048; C07K 2319/00; C12N 2506/11; C12N 2501/998; C12N 2501/73; C12N 2501/04; C12N 2310/20; C12N 2800/80

(57) ABSTRACT

Disclosed herein are compositions and methods for programming immune cell function though targeted gene regulation.

### CROSS REFERENCE TO RELATED APPLICATIONS

This application claims priority to U.S. Provisional Application No. 62/373,343, filed Aug. 10, 2016, which is incorporated herein by reference in its entirety.

### STATEMENT OF GOVERNMENT INTEREST

This invention was made with government support under federal grant numbers 1R01DA036865 and 1DP2-OD008586 awarded by NIH. The U.S. Government has certain rights to this invention.

### SEQUENCE LISTING

The instant application contains a Sequence Listing which has been submitted electronically in ASCII format and is hereby incorporated by reference in its entirety. Said ASCII copy, created on Aug. 10, 2017, is named 028193-9236-WO00 Sequence Listing.txt and is 175,788 bytes in size.

### TECHNICAL FIELD

The present disclosure is directed to compositions and methods for programming immune cell function through targeted gene regulation.

### BACKGROUND

Immunotherapy and regenerative medicine provides the exciting potential for cell-based therapies to treat many diseases and restore damaged tissues, but the inability to precisely control cell function has limited the ultimate success of this field. For over 40 years, gene therapy has been proposed as an approach to cure genetic diseases by adding functional copies of genes to the cells of patients with defined genetic mutations. However, this field has been limited by the available technologies for adding extra genetic material to human genomes. In recent years, the advent of synthetic biology has led to the development of technologies for precisely controlling gene networks that determine cell behavior. Several new technologies have emerged for manipulating genes in their native genomic context by engineering synthetic transcription factors that can be targeted to any DNA sequence. This includes new technologies that have enabled targeted human gene activation and repression, including the engineering of transcription factors based on zinc finger proteins, TALEs, and the CRISPR/Cas9 system.

Regulatory T cells (T_{reg} cells) are a subset of T cells that promote immune tolerance, preventing autoimmune reactions against self-antigens by effector T cells that escape negative selection in the thymus. Native T_{reg} cells arise during normal T cell development in the thymus through medium-affinity interactions between self-antigen and the T cell receptor and can also arise in peripheral tissues depending on the strength and duration of the T cell receptor engagement and a requirement for TGFβ. There remains a need for the ability to precisely regulate any gene as it occurs naturally in the genome, such as the rewiring of genetic circuits to influence immune cell function, as a means to address a variety of diseases and disorders while circumventing some of the traditional challenges of gene therapy.

### SUMMARY

The present invention is directed to a DNA targeting system for programming immune cell function. The DNA targeting system includes a fusion protein and at least one guide RNA (gRNA). The fusion protein includes two heterologous polypeptide domains, wherein the first polypeptide domain includes a Clustered Regularly Interspaced Short Palindromic Repeats associated (Cas) protein and the second polypeptide domain includes a peptide having histone acetyltransferase activity a peptide having transcription activation activity, or a peptide having transcription repressor activity. The at least one gRNA targets a target region in at least one gene of FoxP3, IL2RA, CTLA4, GATA3, RORC, PDCD1, TNFRSF18, CCR7, CCR4, CXCR3, or TBX21.

The present invention is directed to a DNA targeting system for programming immune cell function. The DNA targeting system includes a fusion protein. The fusion protein includes two heterologous polypeptide domains, wherein the first polypeptide domain includes a zinc finger protein, a TAL effector, a meganuclease, or a Clustered Regularly Interspaced Short Palindromic Repeats associated (Cas) protein and the second polypeptide domain includes a peptide having histone acetyltransferase activity, a peptide having transcription activation activity, or a peptide having transcription repressor activity. The at least one gRNA targets a target region in at least one gene of FoxP3, IL2RA, CTLA4, GATA3, RORC, PDCD1, TNFRSF18, CCR7, CCR4, CXCR3, or TBX21.

### BRIEF DESCRIPTION OF THE DRAWINGS

FIG. 1 shows gene positions and DNAse I hypersensitivity tracks for T_{reg}, Tₕ₁₇, Tₕ₁, Tₕ₂ and naive Tₕ₀ cell types. Data was taken from the University of California at San Diego Genome Browser, human genome assembly GRCh37/hg19. Guide RNA molecules were designed to target the regions shaded in red in the upper panel. The lower panel shows the distribution of gRNAs across the intron 1 (CNS2 enhancer element) and promoter DNAse hypersensitivity peaks.

FIG. 2 shows upregulation of FoxP3 by dCas9-p300 in the K562 FoxP3-2A-DsRed reporter cell line. Cells stably expressing dCas9-p300 were electroporated with pooled vectors encoding 5 guide RNAs targeting either the FoxP3 promoter (left panel) or intron 1 (middle panel) DNAse hypersensitive sites. DsRed fluorescence was assessed 48 hours after electroporation and compared to cells transfected with a vector expressing a scrambled gRNA control (shaded black histograms). Cells were transfected in parallel with a GFP expression vector (right panel), which shows that transfection efficiency was >60% using this method.

FIG. 3 shows K562 cells stably expressing dCas9-p300 were transiently transfected with pools of 5 expression vectors for gRNAs targeting the FoxP3 promoter or intron 1 DNAse hypersensitive sites, the combined pool of 10 gRNAs targeting both the promoter and intron 1 DNAse hypersensitive sites, or a GFP control vector. FoxP3 expression in all groups was assessed after 48 hours by direct antibody staining against FoxP3. Increased expression of FoxP3 was observed in cells transfected with gRNAs against the DNAse hypersensitive sites in the promoter, intron 1, or both compared to cells transfected with a control vector expressing a scrambled gRNA (shaded black histograms).

FIG. 4 shows real-time PCR indicating that transfection with expression vectors for 5 gRNAs targeting either the promoter DNAse hypersensitive site or the intron 1 DNAse hypersensitive site increased FoxP3 mRNA by 23-fold (p<0.0001) and 1.9-fold (p<0.01), respectively over cells transfected with a control GFP expression vector. Cells transfected with an expression vector for a scrambled gRNA did not significantly increase FoxP3 mRNA (p>0.1 versus cells transfected with the GFP expression vector).

FIG. 5 shows a schematic of FoxP3 enhancers and their effect on gene regulation.

FIG. 6 shows the amino acid sequence of dCas9^{FL p300} (SEQ ID NO: 25).

FIG. 7 shows the amino acid sequence of dCas9^{p300 Core} (SEQ ID NO: 26).

FIG. 8 shows a schematic of dCas9^{KRAB}.

FIG. 9 shows the amino acid sequence of Nm-dCas9^{p300 Core} (SEQ ID NO: 27).

FIG. 10 shows the logic for automated identification of differential DHSs.

FIG. 11 shows the genome-wide DHSs unique to T_{reg} cells.

FIG. 12 shows the effect of window size on hits.

FIG. 13 shows the genome wide DHSs according to window size.

FIG. 14 shows the effect of selecting window size based on gRNA library size.

FIG. 15 shows the DHS hits near genes relevant to T_{reg} cells, i.e., FOXP3.

FIG. 16 shows the DHS hits near genes relevant to T_{reg} cells, i.e., IL2RA and CTLA4.

FIG. 17 shows the DHS hits near genes relevant to T_{reg} cells, i.e., GATA3 (Tₕ₂) and RORC (Tₕ₁₇).

FIG. 18 shows the DHS hits near genes relevant to T_{reg} cells, i.e., TBX21.

FIGS. 19A-19C show that epigenetic modification of the FOXP3 promoter or an enhancer within intron 1 induces FoxP3 expression in primary human T cells. FIG. 19A shows CD3 and FoxP3 expression of single cells in the preparation for the indicated guide RNA pools. FIG. 19B shows histograms showing FoxP3 expression of CD3-positive cells gated from FIG. 19A. The shaded solid histograms show FoxP3 expression of cells transduced with dCas9-p300 only (no guide RNA). Open histograms show FoxP3 expression of cells transduced with the indicated guide RNA pools. FIG. 19C shows mean fluorescence intensity (MFI) and fold change of each group compared to cells transduced with dCas9-p300 only (no guide) for the populations in FIG. 19B.

FIG. 20 shows gene positions and DNAse I hypersensitivity tracks for Jurkat cells.

FIG. 21 shows real-time PCR indicating that transfection with expression vectors for 5 gRNAs targeting the promoter DNAse hypersensitive site increased CCR7 mRNA as compared to no gRNA and no-targeted gRNA controls.

FIG. 22 shows cells stably expressing dCas9-p300 were transiently transfected with pools of 5 expression vectors for gRNAs targeting the CCR7 promoter DNAse hypersensitive sites, no gRNA control, or non-targeted gRNA control. CCR7 expression in all groups was assessed by direct antibody staining against CCR7. Increased expression of FoxP3 was observed in cells transfected with gRNAs against the DNAse hypersensitive sites in the promoter. Shaded solid histograms show FoxP3 expression of cells transduced with dCas9-p300 only (no guide RNA).

FIG. 23 shows the amino acid sequence of dCas9^{VP64} (SEQ ID NO: 34).

FIG. 24 shows the amino acid sequence of Nm-dCas9^{VP64} (SEQ ID NO: 35).

FIG. 25 shows the amino acid sequence of ICAM1 ZF^{VP} (SEQ ID NO: 36).

FIG. 26 shows the amino acid sequence of ICAM1 ZF^{p3000 Core} (SEQ ID NO: 37).

FIG. 27A shows FoxP3 expression of primary T cells that were mock electroporated (gray), electroporated with synthetic FoxP3 gRNA only (red), or electroporated with synthetic FoxP3 gRNA and in vitro transcribed dCas9-2xVP64 mRNA (SEQ ID NO: 34) (blue).

FIG. 27B shows flow cytometry of T_{reg} specific surface markers CD25 and CD127 showing that Fox-P3-activated primary T cells are reprogrammed to have T_{reg} surface profile of CD25^{hi}, CD127^{lo}.

### DETAILED DESCRIPTION

The present disclosure provides systems and methods for programming immune cell function. In particular, the present disclosure provides an innovative method to rewire cellular gene circuits and created a synthetic transcriptional system in a manner that allows target cells, such as cell lines and/or primary T cells, to be engineered with gene regulatory factors or enhancers to induce differentiation, change immune cell phenotype, and/or reprogram immune cell function. For example, cell lines may be induced to transition primary T cells. The modified cell may be used for immunotherapies, such as CAR-T therapies (e.g., engraftment, durability, and potency) as well as therapies to treat autoimmune disease and cancer.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Cancer” as used herein refers to the uncontrolled and unregulated growth of abnormal cells in the body. Cancerous cells are also called malignant cells. Cancer may invade nearby parts of the body and may also spread to more distant parts of the body through the lymphatic system or bloodstream. Cancers include Adrenocortical Carcinoma, Anal Cancer, Bladder Cancer, Brain Tumor, Breast Cancer, Carcinoid Tumor, Gastrointestinal, Carcinoma of Unknown Primary, Cervical Cancer, Colon Cancer, Endometrial Cancer, Esophageal Cancer, Extrahepatic Bile Duct Cancer, Ewings Family of Tumors (PNET), Extracranial Germ Cell Tumor, Intraocular Melanoma Eye Cancer, Gallbladder Cancer, Gastric Cancer (Stomach), Extragonadal Germ Cell Tumor, Gestational Trophoblastic Tumor, Head and Neck Cancer, Hypopharyngeal Cancer, Islet Cell Carcinoma, Kidney Cancer (renal cell cancer), Laryngeal Cancer, Acute Lymphoblastic Leukemia, Leukemia, Acute Myeloid, Chronic Lymphocytic Leukemia, Chronic Myelogenous Leukemia, Hairy Cell Leukemia, Lip and Oral Cavity Cancer, Liver Cancer, Non-Small Cell Lung Cancer, Small Cell Lung Cancer, AIDS-Related Lymphoma, Central Nervous System (Primary) Lymphoma, Cutaneous T-Cell Lymphoma, Hodgkin's Disease Lymphoma, Non-Hodgkin's Disease Lymphoma, Malignant Mesothelioma, Melanoma, Merkel Cell Carcinoma, Metasatic Squamous Neck Cancer with Occult Primary, Multiple Myeloma and Other Plasma Cell Neoplasms, Mycosis Fungoides, Myelodysplastic Syndrome, Myeloproliferative Disorders, Nasopharyngeal Cancer, euroblastoma, Oral Cancer, Oropharyngeal Cancer, Osteosarcoma, Ovarian Epithelial Cancer, Ovarian Germ Cell Tumor, Pancreatic Cancer, Exocrine, Pancreatic Cancer, Islet Cell Carcinoma, Paranasal Sinus and Nasal Cavity Cancer, Parathyroid Cancer, Penile Cancer, Pituitary Cancer, Plasma Cell Neoplasm, Prostate Cancer, Rhabdomyosarcoma, Rectal Cancer, Renal Cell Cancer (cancer of the kidney), Transitional Cell Renal Pelvis and Ureter, Salivary Gland Cancer, Sezary Syndrome, Skin Cancer, Small Intestine Cancer, Soft Tissue Sarcoma, Testicular Cancer, Malignant Thymoma, Thyroid Cancer, Urethral Cancer, Uterine Cancer, Unusual Cancer of Childhood, Vaginal Cancer, Vulvar Cancer, and Wilms' Tumor.

“Cell therapy” as used herein refers to a therapy in which cellular material is injected into a patient. The cellular material may be intact, living cells. For example, T cells capable of fighting cancer cells via cell-mediated immunity may be injected in the course of immunotherapy. Cell therapy is also called cellular therapy or cytotherapy.

“Chromatin” as used herein refers to an organized complex of chromosomal DNA associated with histones.

“Chronic disease” as used refers to a long-lasting condition that can be controlled but not cured.

“Cis-regulatory elements” or “CREs” as used interchangeably herein refers to regions of non-coding DNA which regulate the transcription of nearby genes. CREs are found in the vicinity of the gene, or genes, they regulate. CREs typically regulate gene transcription by functioning as binding sites for transcription factors. Examples of CREs include promoters and enhancers.

“Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

“Endogenous gene” as used herein refers to a gene that originates from within an organism, tissue, or cell. An endogenous gene is native to a cell, which is in its normal genomic and chromatin context, and which is not heterologous to the cell. Such cellular genes include, e.g., animal genes, plant genes, bacterial genes, protozoal genes, fungal genes, mitochondrial genes, and chloroplastic genes.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Fusion protein” as used herein refers to a chimeric protein created through the joining of two or more genes that originally coded for separate proteins. The translation of the fusion gene results in a single polypeptide with functional properties derived from each of the original proteins.

“FoxP3” or “FOXP3” as used interchangeably here refers to a protein involved in immune system responses. FoxP3, also known as scurfin, is a member of the FOX protein family. FoxP3 appears to function as a master regulator of the regulatory pathway in the development and function of regulatory T cells. Regulatory T cells generally turn the immune response down. In cancer, an excess of regulatory T cell activity can prevent the immune system from destroying cancer cells. In autoimmune disease, a deficiency of regulatory T cell activity can allow other autoimmune cells to attack the body's own tissues. FOX proteins belong to the forkhead/winged-helix family of transcriptional regulators and are presumed to exert control via similar DNA binding interactions during transcription. In regulatory T cell model systems, the FOXP3 transcription factor occupies the promoters for genes involved in regulatory T-cell function, and may repress transcription of key genes following stimulation of T cell receptors. The human FOXP3 genes contain 11 coding exons. Exon-intron boundaries are identical across the coding regions of the mouse and human genes. By genomic sequence analysis, the FOXP3 gene maps to the p arm of the X chromosome (specifically, Xp11.23)

“Genetic construct” as used herein refers to the DNA or RNA molecules that comprise a nucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operably linked to a coding sequence that encodes a protein such that when present in the cell of the individual, the coding sequence will be expressed.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, e.g., a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (e.g., a “fusion protein,” where the two subsequences are encoded by a single nucleic acid sequence).

“Histone acetyltransferases” or “HATs” are used interchangeably herein refers to enzymes that acetylate conserved lysine amino acids on histone proteins by transferring an acetyl group from acetyl CoA to form ε-N-acetyllysine. DNA is wrapped around histones, and, by transferring an acetyl group to the histones, genes can be turned on and off. In general, histone acetylation increases gene expression as it is linked to transcriptional activation and associated with euchromatin. Histone acetyltransferases can also acetylate non-histone proteins, such as nuclear receptors and other transcription factors to facilitate gene expression.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Immunotherapy” as used herein refers to the treatment of disease by inducing, enhancing, or suppressing an immune response. Immunotherapies designed to elicit or amplify an immune response are classified as activation immunotherapies, while immunotherapies that reduce or suppress are classified as suppression immunotherapies.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“p300 protein,” “EP300,” or “E1A binding protein p300” as used interchangeably herein refers to the adenovirus E1A-associated cellular p300 transcriptional co-activator protein encoded by the EP300 gene. p300 is a highly conserved acetyltransferase involved in a wide range of cellular processes. p300 functions as a histone acetyltransferase that regulates transcription via chromatin remodeling and is involved with the processes of cell proliferation and differentiation.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. The subject or patient may be undergoing other forms of treatment.

“Target enhancer” as used herein refers to enhancer that is targeted by a gRNA and DNA targeting system. The target enhancer may be within the target region.

“Target region” as used herein refers to a cis-regulatory region or a trans-regulatory region of a target gene to which the guide RNA is designed to recruit the DNA targeting system to modulate the epigenetic structure and allow the activation of gene expression of the target gene.

“Target regulatory element” as used herein refers to a regulatory element that is targeted by a gRNA and DNA targeting system. The target regulatory element may be within the target region.

“Transcribed region” as used herein refers to the region of DNA that is transcribed into single-stranded RNA molecule, known as messenger RNA, resulting in the transfer of genetic information from the DNA molecule to the messenger RNA. During transcription, RNA polymerase reads the template strand in the 3′ to 5′ direction and synthesizes the RNA from 5′ to 3′. The mRNA sequence is complementary to the DNA strand.

“Transcriptional Start Site” or “TSS” as used interchangeably herein refers to the first nucleotide of a transcribed DNA sequence where RNA polymerase begins synthesizing the RNA transcript.

“Transcriptional repressors” as used herein refers to a DNA- or RNA-binding protein that inhibits the expression of one or more genes by binding to the operator or associated silencers. For example, a DNA-binding repressor blocks the attachment of RNA polymerase to the promoter, thus preventing transcription of the genes into messenger RNA; an RNA-binding repressor binds to the mRNA and prevents translation of the mRNA into protein.

“Trans-regulatory elements” as used herein refers to regions of non-coding DNA which regulate the transcription of genes distant from the gene from which they were transcribed. Trans-regulatory elements may be on the same or different chromosome from the target gene. Trans-regulatory elements may include enhancers of the target gene.

“Treat”, “treating” or “treatment” are each used interchangeably herein to describe reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of an antibody or pharmaceutical composition of the present invention to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

“T cell” or “T lymphocyte” as used interchangeably herein refers to a cell derived from thymus among lymphocytes involved in an immune response.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of +2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid. For example, the vector may encode a DNA targeting system having an amino acid sequence of SEQ ID NO: 25, 26, or 27 and/or at least one gRNA nucleotide sequence of any one of SEQ ID NOs: 11-20 or 43-47.

### 2. DNA TARGETING SYSTEM FOR PROGRAMMING IMMUNE CELL FUNCTION

Provided herein are DNA targeting systems for use in programming immune cell function. In some embodiments, the DNA targeting system can include fusion protein that can be used to program an immune cell. The fusion protein includes two heterologous polypeptide domains, wherein the first polypeptide domain includes a zinc finger protein, a TAL effector (TALE), a meganuclease, or a CRISPR/Cas9, and the second polypeptide domain includes a peptide having histone acetyltransferase activity, a peptide having transcription activation activity, or a peptide having transcription repressor activity. The fusion protein targets a target region in any gene of interest. In some embodiments, the fusion protein includes an amino acid sequence of any one of SEQ ID NO: 27, SEQ ID NO: 28, SEQ ID NO: 29, SEQ ID NO: 34, SEQ ID NO: 35, SEQ ID NO: 36, or SEQ ID NO: 37.

In some embodiment, the DNA targeting system includes a fusion protein and at least one guide RNA (gRNA). The fusion protein includes two heterologous polypeptide domains, wherein the first polypeptide domain includes a Clustered Regularly Interspaced Short Palindromic Repeats associated (Cas) protein and the second polypeptide domain includes a peptide having histone acetyltransferase activity, a peptide having transcription activation activity, or a peptide having transcription repressor activity. The gRNA targets a target region in any gene of interest. In particular, the gene of interest may be involved in the development and function of regulatory T cells, such as FoxP3, IL2RA, CTLA4, GATA3, RORC, PDCD1, TNFRSF18, CCR7, CCR4, CXCR3, or TBX21. In some embodiments, the second polypeptide domain includes a peptide having transcriptional activation or histone acetyltransferase activity and the DNA targeting system is a CRISPR/Cas9-based gene activation system. In some embodiments, the second polypeptide domain includes a peptide having transcription repressor activity and the DNA targeting system is a CRISPR/Cas9-based gene repressor system

a) CRISPR System

The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a ‘memory’ of past exposures. Cas9 forms a complex with the 3′ end of the single guide RNA (“sgRNA”), and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the CRISPR RNA (“crRNA”), i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed chimeric sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex.

An engineered form of the Type II effector system of Streptococcus pyogenes was shown to function in human cells for genome engineering. In this system, the Cas9 protein was directed to genomic target sites by a synthetically reconstituted “guide RNA” (“gRNA”, also used interchangeably herein as a chimeric sgRNA, which is a crRNA-tracrRNA fusion that obviates the need for RNase III and crRNA processing in general.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Type II systems have differing PAM requirements. The S. pyogenes CRISPR system may have the PAM sequence for this Cas9 (SpCas9) as 5′-NRG-3′, where R is either A or G, and characterized the specificity of this system in human cells. A unique capability of the CRISPR/Cas9 system is the straightforward ability to simultaneously target multiple distinct genomic loci by co-expressing a single Cas9 protein with two or more sgRNAs. For example, the Streptococcus pyogenes Type II system naturally prefers to use an “NGG” sequence, where “N” can be any nucleotide, but also accepts other PAM sequences, such as “NAG” in engineered systems (Hsu et al., Nature Biotechnology (2013) doi:10.1038/nbt.2647). Similarly, the Cas9 derived from Neisseria meningitidis (NmCas9) normally has a native PAM of NNNNGATT, but has activity across a variety of PAMs, including a highly degenerate NNNNGNNN PAM (Esvelt et al. Nature Methods (2013) doi:10.1038/nmeth.2681).

b) Cas9

The DNA targeting system may include a Cas9 protein or a Cas9 fusion protein. Cas9 protein is an endonuclease that cleaves nucleic acid and is encoded by the CRISPR loci and is involved in the Type II CRISPR system. The Cas9 protein may be from any bacterial or archaea species, such as Streptococcus pyogenes, Streptococcus thermophiles, or Neisseria meningitides. The Cas9 protein may be mutated so that the nuclease activity is inactivated. In some embodiments, an inactivated Cas9 protein from Streptococcus pyogenes (iCas9, also referred to as “dCas9”; SEQ ID NO: 31) may be used. As used herein, “iCas9” and “dCas9” both refer to a Cas9 protein that has the amino acid substitutions D10A and H840A and has its nuclease activity inactivated. In some embodiments, an inactivated Cas9 protein from Neisseria meningitides, such as NmCas9 having an amino acid sequence of SEQ ID NO: 32, may be used.

c) CRISPR/Cas9-Based Gene Activation System

The CRISPR/Cas9-based gene activation systems can be used to activate gene expression of a target gene that is involved in development and function of regulatory T cells. In some embodiments, the CRISPR/Cas9-based gene activation system includes a fusion protein of a Cas9 protein that does not have nuclease activity, such as dCas9, and a transactivation domain. In some embodiments, the CRISPR/Cas9-based gene activation system includes a fusion protein of a Cas9 protein that does not have nuclease activity, such as dCas9, and a histone acetyltransferase or histone acetyltransferase effector domain. Histone acetylation, carried out by histone acetyltransferases (HATs), plays a fundamental role in regulating chromatin dynamics and transcriptional regulation. The histone acetyltransferase protein releases DNA from its heterochromatin state and allows for continued and robust gene expression by the endogenous cellular machinery. The recruitment of an acetyltransferase by dCas9 to a genomic target site may directly modulate epigenetic structure.

The CRISPR/Cas9-based gene activation system may catalyze acetylation of histone H3 lysine 27 at its target sites, leading to robust transcriptional activation of target genes from promoters and proximal and distal enhancers. The CRISPR/Cas9-based gene activation system is highly specific and may be guided to the target gene using as few as one guide RNA. The CRISPR/Cas9-based gene activation system may activate the expression of one gene or a family of genes by targeting enhancers at distant locations in the genome.

i) Histone Acetyltransferase (HAT) Protein

The CRISPR/Cas9-based gene activation system may include a histone acetyltransferase protein, such as a p300 protein, CREB binding protein (CBP; an analog of p300), GCN5, or PCAF, or fragment thereof. The p300 protein regulates the activity of many genes in tissues throughout the body. The p300 protein plays a role in regulating cell growth and division, prompting cells to mature and assume specialized functions (differentiate) and preventing the growth of cancerous tumors. The p300 protein may activate transcription by connecting transcription factors with a complex of proteins that carry out transcription in the cell's nucleus. The p300 protein also functions as a histone acetyltransferase that regulates transcription via chromatin remodeling.

The histone acetyltransferase protein may include a human p300 protein or a fragment thereof. The histone acetyltransferase protein may include a wild-type human p300 protein or a mutant human p300 protein, or fragments thereof. The histone acetyltransferase protein may include the core lysine-acetyltransferase domain of the human p300 protein, i.e., the p300 HAT Core (also known as “p300 Core”). In some embodiments, the histone acetyltransferase protein includes an amino acid sequence of SEQ ID NO: 22 or 23.

The CRISPR/Cas9-based gene activation system may include a histone acetylation effector domain. The histone acetylation effector domain may be the catalytic histone acetyltransferase (HAT) core domain of the human E1A-associated protein p300 (also referred to herein as “p300 Core”). In some embodiments, the p300 Core includes amino acids 1048-1664 of SEQ ID NO: 23 (i.e., SEQ ID NO: 24). In some embodiments, the CRISPR/Cas9-based gene activation system includes a dCas9^{p300 Core} fusion protein of SEQ ID NO: 26 or an Nm-dCas9^{p300 Core} fusion protein of SEQ ID NO: 27. The p300 Core acetylates lysine 27 on histone H3 (H3K27ac) and may provide H3K27ac enrichment.

The dCas9^{p300 Core} fusion protein is a potent and easily programmable tool to synthetically manipulate acetylation at targeted endogenous loci, leading to regulation of proximal and distal enhancer-regulated genes. The fusion of the catalytic core domain of p300 to dCas9 may result in substantially higher transactivation of downstream genes than the direct fusion of full-length p300 protein despite robust protein expression. The dCas9^{p300 Core} fusion protein may also exhibit an increased transactivation capacity relative to dCas9^{VP64}, including in the context of the Nm-dCas9 scaffold, especially at distal enhancer regions, at which dCas9^{VP64} displayed little, if any, measurable downstream transcriptional activity. Additionally, the dCas9^{p300} core displays precise and robust genome-wide transcriptional specificity. dCas9^{p300} Core may be capable of potent transcriptional activation and co-enrichment of acetylation at promoters targeted by the epigenetically modified enhancer.

The dCas9^{p300 Core} may activate gene expression through a single gRNA that target and bind a promoters and/or a characterized enhancer. This technology also affords the ability to synthetically transactivate distal genes from putative and known regulatory regions and simplifies transactivation via the application of a single programmable effector and single target site. These capabilities allow multiplexing to target several promoters and/or enhancers simultaneously. The mammalian origin of p300 may provide advantages over virally-derived effector domains for in vivo applications by minimizing potential immunogenicity.

ii) Transcription Activation Activity

The CRISPR/Cas9-based gene activation system may include a transactivation domain. The second polypeptide domain may have transcription activation activity, i.e., a transactivation domain. For example, gene expression of endogenous mammalian genes, such as human genes, may be achieved by targeting a fusion protein of iCas9 and a transactivation domain to mammalian promoters via combinations of gRNAs. The transactivation domain may include a VP16 protein, multiple VP16 proteins, such as a VP48 domain or VP64 domain, or p65 domain of NF kappa B transcription activator activity. For example, the fusion protein may be iCas9-VP64. In some embodiments, the fusion protein may be dCas9^{VP64} (SEQ ID NO: 34) or Nm-dCas9^{P64} (SEQ ID NO: 35).

iii) Methylase Activity

The CRISPR/Cas9-based gene activation system may include a methylase activity domain. The second polypeptide domain may have methylase activity, which involves transferring a methyl group to DNA, RNA, protein, small molecule, cytosine or adenine. The second polypeptide domain may include a DNA methyltransferase. In some embodiments, the methylase activity domain is DNA (cytosine-5)-methyltransferase 3A (DNMT3a). DNMT3a is an enzyme that catalyzes the transfer of methyl groups to specific CpG structures in DNA. The enzyme is encoded in humans by the DNMT3A gene.

d) CRISPR/Cas9-Based Gene Repressor System

The CRISPR/Cas9-based gene repressor systems can be used to repress gene expression of a target gene that is involved in development and function of regulatory T cells. The CRISPR/Cas9-based gene repressor system includes a fusion protein of a Cas9 protein that does not have nuclease activity, such as dCas9, and a transcriptional repressor effector domain. The recruitment of a transcriptional repressor protein by dCas9 to a genomic target site may directly modulate epigenetic structure. The CRISPR/Cas9-based gene repressor system is highly specific and may be guided to the target gene using as few as one guide RNA. The CRISPR/Cas9-based gene transcriptional repressor system may repress the expression of one gene or a family of genes by targeting enhancers at distant locations in the genome.

i) Transcriptional Repression Domain

The CRISPR/Cas9-based gene repressor system may include a transcriptional repression domain, such as a Kruppel associated box (KRAB) domain, or fragment thereof. The KRAB domain is present in approximately 400 human zinc finger protein-based transcription factors (KRAB zinc finger proteins). The KRAB domain typically consists of about 75 amino acid residues, while the minimal repression module is approximately 45 amino acid residues. The KRAB domain may function through protein-protein interactions via two amphipathic helices. The transcriptional repression domain may include a human KRAB domain or a fragment thereof. The transcriptional repression domain may include a wild-type human KRAB domain or a mutant human KRAB domain, or fragments thereof. In some embodiments, the CRISPR/Cas9-based gene repressor system includes a dCas9^{AB} fusion protein (see e.g., FIG. 9).

ii) Demethylase Activity

The CRISPR/Cas9-based gene repressor system may include a demethylase activity domain. The second polypeptide domain may include an enzyme that remove methyl (CH3-) groups from nucleic acids, proteins (in particular histones), and other molecules. Alternatively, the second polypeptide may covert the methyl group to hydroxymethylcytosine in a mechanism for demethylating DNA. The second polypeptide may catalyze this reaction. For example, the second polypeptide that catalyzes this reaction may be Ten-eleven translocation methylcytosine dioxygenase 1 (Tet1) or Lysine-specific histone demethylase 1 (LSD1).

TET1 is a member of the TET family of enzymes that in humans is encoded by the TET1 gene. TET1 catalyzes the conversion of the modified DNA base 5-methylcytosine (5-mC) to 5-hydroxymethylcytosine (5-hmC) and produces 5-hmC by oxidation of 5-mC in an iron and alpha-ketoglutarate dependent manner. The conversion of 5-mC to 5-hmC may be the initial step of active DNA demethylation in mammals. Additionally, downgrading TET1 has decreased levels of 5-formylcytosine (5-fC) and 5-carboxylcytosine (5-caC) in both cell cultures and mice. TET1 may facilitate nuclear reprogramming of somatic cells to iPS cells.

LSD1; also known as lysine-specific histone demethylase 1A (KDM1A) and lysine (K)-specific demethylase 1A, is a protein in humans that is encoded by the KDM1A gene. LSD1 is a flavin-dependent monoamine oxidase, which can demethylate mono- and di-methylated lysines, specifically histone 3, lysines 4 and 9 (H3K4 and H3K9). This enzyme can have roles critical in embryogenesis and tissue-specific differentiation, as well as oocyte growth. KDM1A may play an important role in the epigenetic “reprogramming” that occurs when sperm and egg come together to make a zygote.

e) gRNA

The DNA targeting systems may include at least one gRNA that targets a nucleic acid sequence. The gRNA provides the targeting of the DNA targeting systems. The gRNA is a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9.

The gRNA may target and bind a target region of a target gene. The target region may be a cis-regulatory region or trans-regulatory region of a target gene. In some embodiments, the target region is a distal or proximal cis-regulatory region of the target gene. In some embodiments, the target region is a distal or proximal trans-regulatory region of the target gene. The gRNA may target and bind a cis-regulatory region or trans-regulatory region of a target gene. In some embodiments, the gRNA may target and bind an enhancer region, a promoter region, or a transcribed region of a target gene. For example, the gRNA may target and bind the target region of at least one of FoxP3, IL2RA, CTLA4, GATA3, RORC, PDCD1, TNFRSF18, CCR7, CCR4, CXCR3, or TBX21.

The target region may include a target enhancer or a target regulatory element. In some embodiments, the target enhancer or target regulatory element controls the gene expression of several target genes. In some embodiments, the target enhancer or target regulatory element controls a cell phenotype that involves the gene expression of one or more target genes. In some embodiments, the identity of one or more of the target genes is known. In some embodiments, the identity of one or more of the target genes is unknown.

In some embodiments, at least one gRNA may target and bind a target region. In some embodiments, between 1 and 20 gRNAs may be used to activate or repress a target gene. For example, between 1 gRNA and 20 gRNAs, between 1 gRNA and 15 gRNAs, between 1 gRNA and 10 gRNAs, between 1 gRNA and 5 gRNAs, between 2 gRNAs and 20 gRNAs, between 2 gRNAs and 15 gRNAs, between 2 gRNAs and 10 gRNAs, between 2 gRNAs and 5 gRNAs, between 5 gRNAs and 20 gRNAs, between 5 gRNAs and 15 gRNAs, or between 5 gRNAs and 10 gRNAs are activated by at least one gRNA. In some embodiments, at least 1 gRNA, at least 2 gRNAs, at least 3 gRNAs, at least 4 gRNAs, at least 5 gene, at least 6 gRNAs, at least 7 gRNAs, at least 8 gRNAs, at least 9 gene, at least 10 gRNAs, at least 11 gRNAs, at least 12 gRNAs, at least 13 gene, at least 14 gRNAs, at least 15 gRNAs, or at least 20 gRNAs may be included in the DNA targeting system.

The DNA targeting system may activate or repress genes at both proximal and distal locations relative the transcriptional start site (TSS). The DNA targeting system may target a region that is at least about 1 base pair to about 100,000 base pairs, at least about 100 base pairs to about 100,000 base pairs, at least about 250 base pairs to about 100,000 base pairs, at least about 500 base pairs to about 100,000 base pairs, at least about 1,000 base pairs to about 100,000 base pairs, at least about 2,000 base pairs to about 100,000 base pairs, at least about 5,000 base pairs to about 100,000 base pairs, at least about 10,000 base pairs to about 100,000 base pairs, at least about 20,000 base pairs to about 100,000 base pairs, at least about 50,000 base pairs to about 100,000 base pairs, at least about 75,000 base pairs to about 100,000 base pairs, at least about 1 base pair to about 75,000 base pairs, at least about 100 base pairs to about 75,000 base pairs, at least about 250 base pairs to about 75,000 base pairs, at least about 500 base pairs to about 75,000 base pairs, at least about 1,000 base pairs to about 75,000 base pairs, at least about 2,000 base pairs to about 75,000 base pairs, at least about 5,000 base pairs to about 75,000 base pairs, at least about 10,000 base pairs to about 75,000 base pairs, at least about 20,000 base pairs to about 75,000 base pairs, at least about 50,000 base pairs to about 75,000 base pairs, at least about 1 base pair to about 50,000 base pairs, at least about 100 base pairs to about 50,000 base pairs, at least about 250 base pairs to about 50,000 base pairs, at least about 500 base pairs to about 50,000 base pairs, at least about 1,000 base pairs to about 50,000 base pairs, at least about 2,000 base pairs to about 50,000 base pairs, at least about 5,000 base pairs to about 50,000 base pairs, at least about 10,000 base pairs to about 50,000 base pairs, at least about 20,000 base pairs to about 50,000 base pairs, at least about 1 base pair to about 25,000 base pairs, at least about 100 base pairs to about 25,000 base pairs, at least about 250 base pairs to about 25,000 base pairs, at least about 500 base pairs to about 25,000 base pairs, at least about 1,000 base pairs to about 25,000 base pairs, at least about 2,000 base pairs to about 25,000 base pairs, at least about 5,000 base pairs to about 25,000 base pairs, at least about 10,000 base pairs to about 25,000 base pairs, at least about 20,000 base pairs to about 25,000 base pairs, at least about 1 base pair to about 10,000 base pairs, at least about 100 base pairs to about 10,000 base pairs, at least about 250 base pairs to about 10,000 base pairs, at least about 500 base pairs to about 10,000 base pairs, at least about 1,000 base pairs to about 10,000 base pairs, at least about 2,000 base pairs to about 10,000 base pairs, at least about 5,000 base pairs to about 10,000 base pairs, at least about 1 base pair to about 5,000 base pairs, at least about 100 base pairs to about 5,000 base pairs, at least about 250 base pairs to about 5,000 base pairs, at least about 500 base pairs to about 5,000 base pairs, at least about 1,000 base pairs to about 5,000 base pairs, or at least about 2,000 base pairs to about 5,000 base pairs upstream from the TSS. The DNA targeting system may target a region that is at least about 1 base pair, at least about 100 base pairs, at least about 500 base pairs, at least about 1,000 base pairs, at least about 1,250 base pairs, at least about 2,000 base pairs, at least about 2,250 base pairs, at least about 2,500 base pairs, at least about 5,000 base pairs, at least about 10,000 base pairs, at least about 11,000 base pairs, at least about 20,000 base pairs, at least about 30,000 base pairs, at least about 46,000 base pairs, at least about 50,000 base pairs, at least about 54,000 base pairs, at least about 75,000 base pairs, or at least about 100,000 base pairs upstream from the TSS.

The DNA targeting system may target a region that is at least about 1 base pair to at least about 500 base pairs, at least about 1 base pair to at least about 250 base pairs, at least about 1 base pair to at least about 200 base pairs, at least about 1 base pair to at least about 100 base pairs, at least about 50 base pairs to at least about 500 base pairs, at least about 50 base pairs to at least about 250 base pairs at least about 50 base pairs to at least about 200 base pairs, at least about 50 base pairs to at least about 100 base pairs, at least about 100 base pairs to at least about 500 base pairs, at least about 100 base pairs to at least about 250 base pairs, or at least about 100 base pairs to at least about 200 base pairs downstream from the TSS. The DNA targeting system may target a region that is at least about 1 base pair, at least about 2 base pairs, at least about 3 base pairs, at least about 4 base pairs, at least about 5 base pairs, at least about 10 base pairs, at least about 15 base pairs, at least about 20 base pairs, at least about 25 base pairs, at least about 30 base pairs, at least about 40 base pairs, at least about 50 base pairs, at least about 60 base pairs, at least about 70 base pairs, at least about 80 base pairs, at least about 90 base pairs, at least about 100 base pairs, at least about 110 base pairs, at least about 120, at least about 130, at least about 140 base pairs, at least about 150 base pairs, at least about 160 base pairs, at least about 170 base pairs, at least about 180 base pairs, at least about 190 base pairs, at least about 200 base pairs, at least about 210 base pairs, at least about 220, at least about 230, at least about 240 base pairs, or at least about 250 base pairs downstream from the TSS.

In some embodiments, the DNA targeting system may target and bind a target region that is on the same chromosome as the target gene but more than 100,000 base pairs upstream or more than 250 base pairs downstream from the TSS. In some embodiments, the DNA targeting system may target and bind a target region that is on a different chromosome from the target gene.

The DNA targeting system may use gRNA of varying sequences and lengths. The gRNA may comprise a complementary polynucleotide sequence of the target DNA sequence followed by NGG. The gRNA may comprise a “G” at the 5′ end of the complementary polynucleotide sequence. The gRNA may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by NGG. The gRNA may target at least one of the promoter region, the enhancer region or the transcribed region of the target gene. The gRNA may include a nucleic acid sequence of at least one of SEQ ID NOs: 11-20 or 43-47.

The DNA targeting system may include at least 1 gRNA, at least 2 different gRNAs, at least 3 different gRNAs at least 4 different gRNAs, at least 5 different gRNAs, at least 6 different gRNAs, at least 7 different gRNAs, at least 8 different gRNAs, at least 9 different gRNAs, or at least 10 different gRNAs. The DNA targeting system may include between at least 1 gRNA to at least 10 different gRNAs, at least 1 gRNA to at least 8 different gRNAs, at least 1 gRNA to at least 4 different gRNAs, at least 2 gRNA to at least 10 different gRNAs, at least 2 gRNA to at least 8 different gRNAs, at least 2 different gRNAs to at least 4 different gRNAs, at least 4 gRNA to at least 10 different gRNAs, or at least 4 different gRNAs to at least 8 different gRNAs.

### 3. TARGET GENES

The DNA targeting system can be designed to target and activate the expression of any target gene or gene of interest that is involved in the development and function of regulatory T cells. The target gene may be an endogenous gene or a transgene. In some embodiments, the target region is located on a different chromosome as the target gene. In some embodiments, the DNA targeting system may include more than 1 gRNA. In some embodiments, the DNA targeting system may include more than 1 different gRNAs. In some embodiments, the different gRNAs bind to different target regions. For example, the different gRNAs may bind to target regions of different target genes and the expression of two or more target genes are activated. Alternatively, the different gRNAs may bind to target regions of the same target gene and the expression of the target gene is activated or repressed. In some embodiments, the target gene may be FoxP3, IL2RA, CTLA4, GATA3, RORC, PDCD1, TNFRSF18, CCR7, CCR4, CXCR3, or TBX21.

In some embodiments, the target gene is a transgene. For example, the target gene may be a chimeric antigen receptor.

a) FoxP3

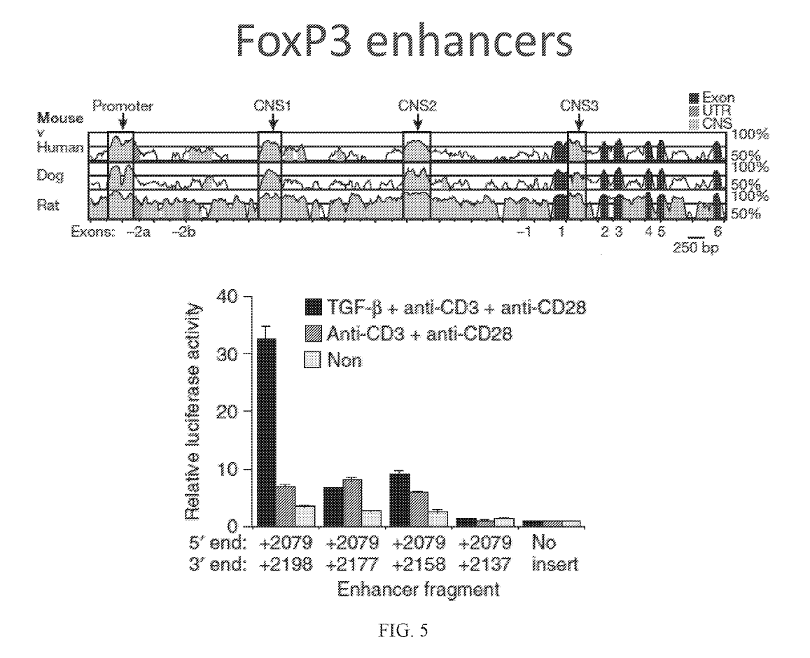

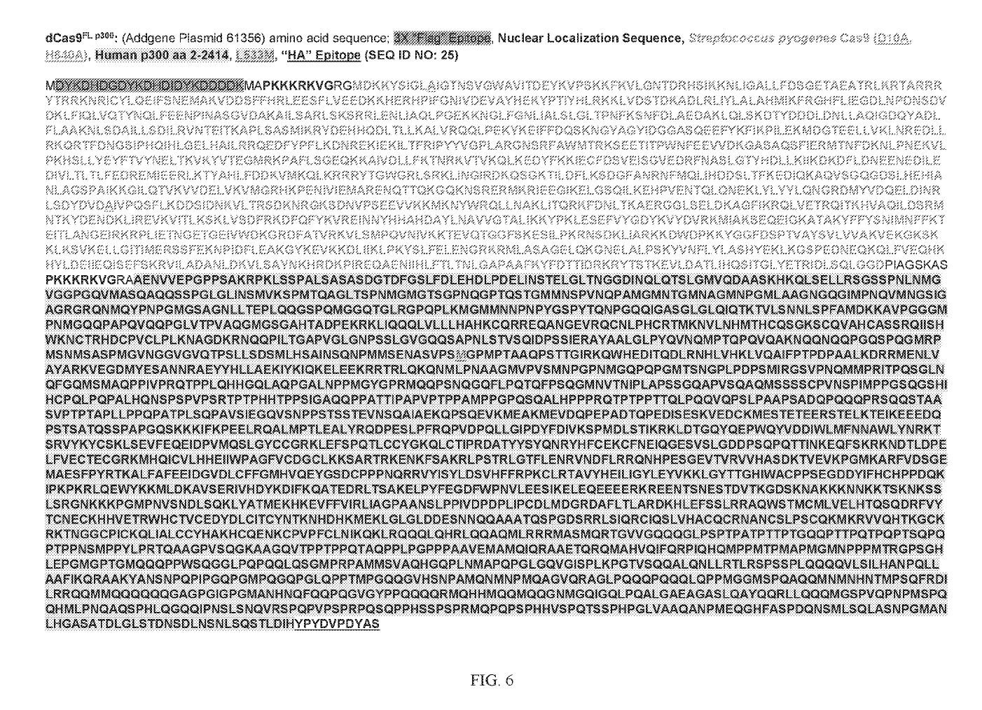

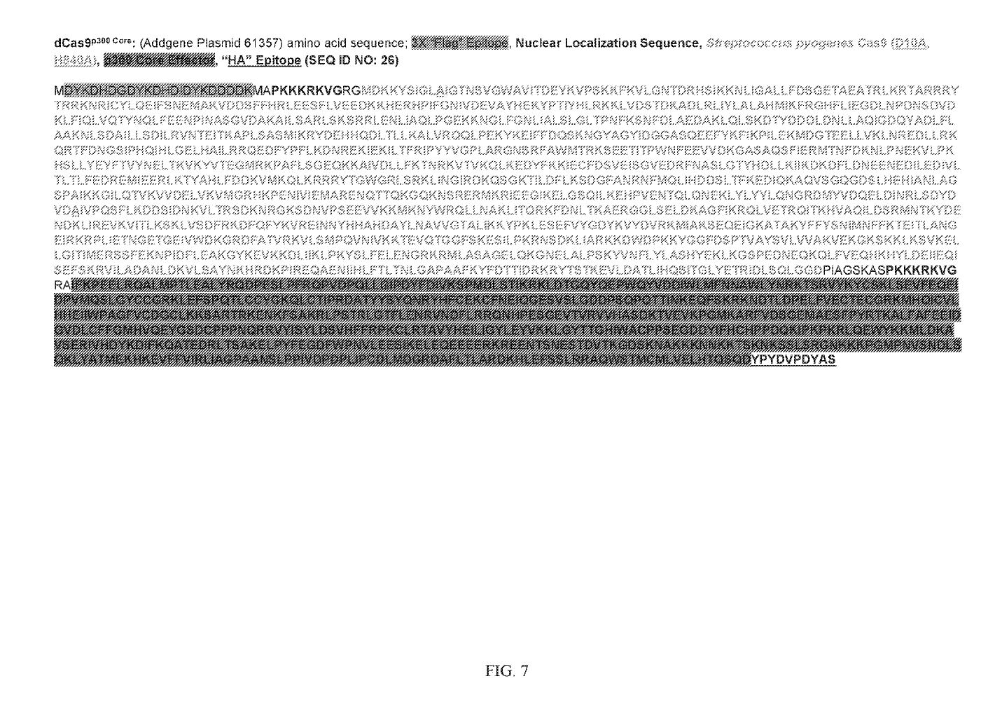

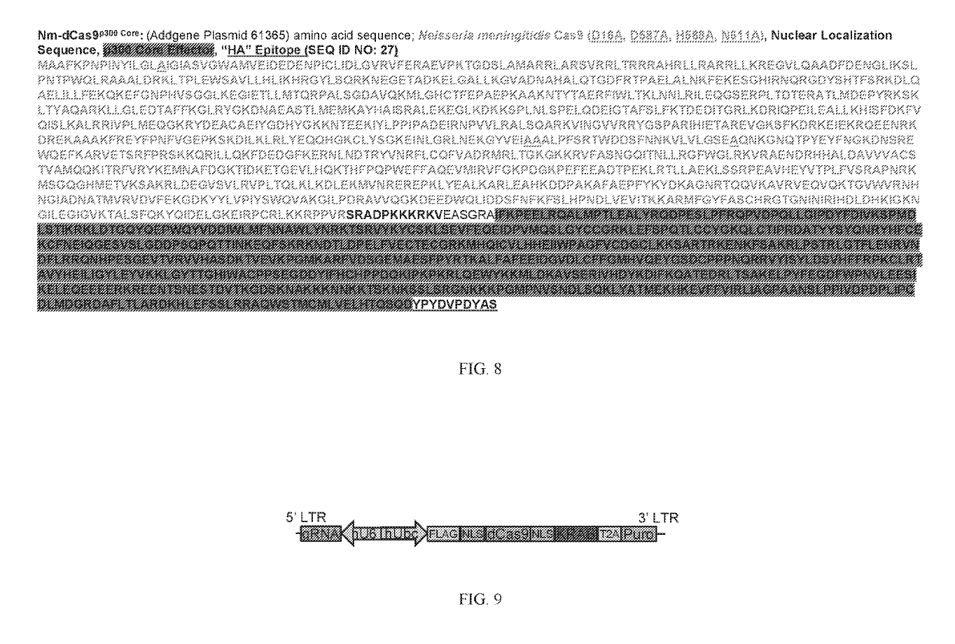

FoxP3 is a transcription factor that is expressed by regulatory T cells and is required for these cells to exert their immunosuppressive effects. FoxP3 is the master transcription factor defining T_{reg} cells. FoxP3 expression is known to be controlled by several enhancers that are responsible for de novo FoxP3 expression during development in the thymus and sustained FoxP3 expression in the periphery. However, specifically modulating the activities of these enhancers has not been possible to date. The DNA targeting system may be used to modulate enhancer accessibility and control of FoxP3 expression. This system can be used to modify genes that influence T cell differentiation and function. In some embodiments, the DNA targeting system targets a FoxP3 enhancer, such as those shown in FIG. 5. For example, the guide RNAs may target the FoxP3 promoter region and the intron 1 enhancer with a nuclease-deficient version of Cas9 (dCas9) fused to the p300 core protein. This strategy specifically directs the acetyltransferase function of the p300 effector towards histones within the FoxP3 promoter or enhancer regions, thereby promoting transcription factor binding that increases FoxP3 gene expression.

Expression levels of FoxP3—the master transcription factor responsible for differentiation of the T_{reg} lineage—can be increased by targeting an epigenetic regulatory protein (p300 histone acetyltransferase) to DNAse hypersensitive regions in the FOXP3 promoter and in the CNS2 enhancer element of intron 1. This strategy can be applied to activate multiple promoter/enhancer elements simultaneously to drive FoxP3 expression in naive primary T cells to generate cells that can maintain a durable immunosuppressive phenotype characteristic of T_{reg} cells. Because of the importance of T_{reg} cells in preventing autoimmune disorders, the epigenetic approach enables the development of cell-based therapies for the treatment of a variety of diseases. The CNS2 enhancer element is responsible for heritable FoxP3 expression. The repression of FoxP3 can suppress T cell formation and may enhance cancer immunotherapy.

In some embodiments, the DNA targeting system targets any target region that modulates FoxP3 expression, such as promoters and enhancers that modulate FoxP3 expression. In some embodiments, the DNA targeting system activates FoxP3 expression. In some embodiments, the DNA targeting represses FoxP3 expression.

### 4. COMPOSITIONS FOR GENE ACTIVATION OR REPRESSION

The present invention is directed to a composition for programming immune cell function. The composition may include the DNA targeting system, as disclosed above. The composition may also include a viral delivery system. For example, the viral delivery system may include an adeno-associated virus vector or a modified lentiviral vector.

Methods of introducing a nucleic acid into a host cell are known in the art, and any known method can be used to introduce a nucleic acid (e.g., an expression construct) into a cell. Suitable methods include, include e.g., viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery.

a) Constructs and Plasmids

The compositions, as described above, may comprise genetic constructs that encodes the DNA targeting system, as disclosed herein. The genetic construct, such as a plasmid or expression vector, may comprise a nucleic acid that encodes the DNA targeting system (such as the CRISPR/Cas9-based acetyltransferase, CRISPR/Cas9-based transcriptional activator, or the CRISPR/Cas9-based transcriptional repressor) and/or at least one of the gRNAs. The compositions, as described above, may comprise genetic constructs that encodes the modified Adeno-associated virus (AAV) vector and a nucleic acid sequence that encodes the DNA targeting system, as disclosed herein. In some embodiments, the compositions, as described above, may comprise genetic constructs that encodes the modified adenovirus vector and a nucleic acid sequence that encodes the DNA targeting system, as disclosed herein. The genetic construct, such as a plasmid, may comprise a nucleic acid that encodes the DNA targeting system. The compositions, as described above, may comprise genetic constructs that encodes a modified lentiviral vector. The genetic construct, such as a plasmid, may comprise a nucleic acid that encodes the CRISPR/Cas9-based acetyltransferase and at least one sgRNA, a nucleic acid that encodes the CRISPR/Cas9-based transcriptional activator, or a nucleic acid that encodes the CRISPR/Cas9-based transcriptional repressor and at least one sgRNA. The genetic construct may be present in the cell as a functioning extrachromosomal molecule. The genetic construct may be a linear minichromosome including centromere, telomeres or plasmids or cosmids.

The genetic construct may also be part of a genome of a recombinant viral vector, including recombinant lentivirus, recombinant adenovirus, and recombinant adenovirus associated virus. The genetic construct may be part of the genetic material in attenuated live microorganisms or recombinant microbial vectors which live in cells. The genetic constructs may comprise regulatory elements for gene expression of the coding sequences of the nucleic acid. The regulatory elements may be a promoter, an enhancer, an initiation codon, a stop codon, or a polyadenylation signal.

The nucleic acid sequences may make up a genetic construct that may be a vector. The vector may be capable of expressing the fusion protein, such as the DNA targeting system, in the cell of a mammal. The vector may be recombinant. The vector may comprise heterologous nucleic acid encoding the fusion protein, such as the DNA targeting system. The vector may be a plasmid. The vector may be useful for transfecting cells with nucleic acid encoding the DNA targeting system, which the transformed host cell is cultured and maintained under conditions wherein expression of the DNA targeting system takes place.

Coding sequences may be optimized for stability and high levels of expression. In some instances, codons are selected to reduce secondary structure formation of the RNA such as that formed due to intramolecular bonding.

The vector may comprise heterologous nucleic acid encoding the DNA targeting system and may further comprise an initiation codon, which may be upstream of the DNA targeting system coding sequence, and a stop codon, which may be downstream of the DNA targeting system coding sequence. The initiation and termination codon may be in frame with the DNA targeting system coding sequence. The vector may also comprise a promoter that is operably linked to the DNA targeting system coding sequence. The DNA targeting system may be under the light-inducible or chemically inducible control to enable the dynamic control of gene activation in space and time. The promoter operably linked to the DNA targeting system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. The promoter may also be a tissue specific promoter, such as a muscle or skin specific promoter, natural or synthetic. Examples of such promoters are described in US Patent Application Publication No. US20040175727, the contents of which are incorporated herein in its entirety.

The vector may also comprise a polyadenylation signal, which may be downstream of the DNA targeting system. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, Calif.).

The vector may also comprise an enhancer upstream of the DNA targeting system or sgRNAs. The enhancer may be necessary for DNA expression. The enhancer may be human actin, human myosin, human hemoglobin, human muscle creatine or a viral enhancer such as one from CMV, HA, RSV or EBV. Polynucleotide function enhancers are described in U.S. Pat. Nos. 5,593,972, 5,962,428, and WO94/016737, the contents of each are fully incorporated by reference. The vector may also comprise a mammalian origin of replication in order to maintain the vector extrachromosomally and produce multiple copies of the vector in a cell. The vector may also comprise a regulatory sequence, which may be well suited for gene expression in a mammalian or human cell into which the vector is administered. The vector may also comprise a reporter gene, such as green fluorescent protein (“GFP”) and/or a selectable marker, such as hygromycin (“Hygro”).

The vector may be expression vectors or systems to produce protein by routine techniques and readily available starting materials including Sambrook et al., Molecular Cloning and Laboratory Manual, Second Ed., Cold Spring Harbor (1989), which is incorporated fully by reference. In some embodiments the vector may comprise the nucleic acid sequence encoding the CRISPR/Cas9-based gene activation system, including the nucleic acid sequence encoding the CRISPR/Cas9-based acetyltransferase and the nucleic acid sequence encoding the at least one gRNA comprising the nucleic acid sequence of at least one of SEQ ID NOs: 11-20 or 43-47.

In some embodiments, the compositions are delivered by mRNA and protein/RNA complexes (Ribonucleoprotein (RNP)). For example, the purified Cas9 protein can be combined with guide RNA to form an RNP complex.

b) Modified Lentiviral Vector

The compositions for gene activation or repression may include a modified lentiviral vector. The modified lentiviral vector includes a first polynucleotide sequence encoding a DNA targeting system and a second polynucleotide sequence encoding at least one sgRNA. The first polynucleotide sequence may be operably linked to a promoter. The promoter may be a constitutive promoter, an inducible promoter, a repressible promoter, or a regulatable promoter.

The second polynucleotide sequence encodes at least 1 sgRNA. For example, the second polynucleotide sequence may encode at least 1 sgRNA, at least 2 sgRNAs, at least 3 sgRNAs, at least 4 sgRNAs, at least 5 sgRNAs, at least 6 sgRNAs, at least 7 sgRNAs, at least 8 sgRNAs, at least 9 sgRNAs, at least 10 sgRNAs, at least 11 sgRNA, at least 12 sgRNAs, at least 13 sgRNAs, at least 14 sgRNAs, at least 15 sgRNAs, at least 16 sgRNAs, at least 17 sgRNAs, at least 18 sgRNAs, at least 19 sgRNAs, at least 20 sgRNAs, at least 25 sgRNA, at least 30 sgRNAs, at least 35 sgRNAs, at least 40 sgRNAs, at least 45 sgRNAs, or at least 50 sgRNAs. The second polynucleotide sequence may encode between 1 sgRNA and 50 sgRNAs, between 1 sgRNA and 45 sgRNAs, between 1 sgRNA and 40 sgRNAs, between 1 sgRNA and 35 sgRNAs, between 1 sgRNA and 30 sgRNAs, between 1 sgRNA and 25 different sgRNAs, between 1 sgRNA and 20 sgRNAs, between 1 sgRNA and 16 sgRNAs, between 1 sgRNA and 8 different sgRNAs, between 4 different sgRNAs and 50 different sgRNAs, between 4 different sgRNAs and 45 different sgRNAs, between 4 different sgRNAs and 40 different sgRNAs, between 4 different sgRNAs and 35 different sgRNAs, between 4 different sgRNAs and 30 different sgRNAs, between 4 different sgRNAs and 25 different sgRNAs, between 4 different sgRNAs and 20 different sgRNAs, between 4 different sgRNAs and 16 different sgRNAs, between 4 different sgRNAs and 8 different sgRNAs, between 8 different sgRNAs and 50 different sgRNAs, between 8 different sgRNAs and 45 different sgRNAs, between 8 different sgRNAs and 40 different sgRNAs, between 8 different sgRNAs and 35 different sgRNAs, between 8 different sgRNAs and 30 different sgRNAs, between 8 different sgRNAs and 25 different sgRNAs, between 8 different sgRNAs and 20 different sgRNAs, between 8 different sgRNAs and 16 different sgRNAs, between 16 different sgRNAs and 50 different sgRNAs, between 16 different sgRNAs and 45 different sgRNAs, between 16 different sgRNAs and 40 different sgRNAs, between 16 different sgRNAs and 35 different sgRNAs, between 16 different sgRNAs and 30 different sgRNAs, between 16 different sgRNAs and 25 different sgRNAs, or between 16 different sgRNAs and 20 different sgRNAs. Each of the polynucleotide sequences encoding the different sgRNAs may be operably linked to a promoter. The promoters that are operably linked to the different sgRNAs may be the same promoter. The promoters that are operably linked to the different sgRNAs may be different promoters. The promoter may be a constitutive promoter, an inducible promoter, a repressible promoter, or a regulatable promoter. At least one sgRNA may bind to a target gene or loci. If more than one sgRNA is included, each of the sgRNAs binds to a different target region within one target loci or each of the sgRNA binds to a different target region within different gene loci.

c) Adeno-Associated Virus Vectors

AAV may be used to deliver the compositions to the cell using various construct configurations. For example, AAV may deliver DNA targeting system and gRNA expression cassettes on separate vectors. Alternatively, if the small Cas9 proteins, derived from species such as Staphylococcus aureus or Neisseria meningitidis, are used then both the Cas9 and up to two gRNA expression cassettes may be combined in a single AAV vector within the 4.7 kb packaging limit.

The composition, as described above, includes a modified adeno-associated virus (AAV) vector. The modified AAV vector may be capable of delivering and expressing the site-specific nuclease in the cell of a mammal. For example, the modified AAV vector may be an AAV-SASTG vector (Piacentino et al. (2012) Human Gene Therapy 23:635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5 and AAV/SASTG vectors that efficiently transduce skeletal muscle or cardiac muscle by systemic and local delivery (Seto et al. Current Gene Therapy (2012) 12:139-151).

### 5. METHODS OF MODULATING T CELL DIFFERENTIATION AND/OR IMMUNE CELL FUNCTION

The present disclosure provides a mechanism for modulating T cell differentiation and/or immune cell function. In some embodiments, the DNA targeting system that includes the CRISPR/Cas9-based gene activation system may be used to activate gene expression of a target gene that is involved in development and function of regulatory T cells. In some embodiments, the DNA targeting system that includes the CRISPR/Cas9-based gene repressor system may be used to repress gene expression of a target gene that is involved in development and function of regulatory T cells. In some embodiments, the target cell, such as a primary T cell, may be modulated to have an immunosuppressive phenotype.

### 6. PHARMACEUTICAL COMPOSITIONS

The DNA targeting system may be in a pharmaceutical composition. The pharmaceutical composition may comprise about 1 ng to about 10 mg of DNA encoding the DNA targeting system. The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The pharmaceutical composition containing the DNA targeting system may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and more preferably, the poly-L-glutamate is present in the pharmaceutical composition containing the DNA targeting system at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the DNA targeting system may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid.

### 7. METHODS OF DELIVERY

Provided herein is a method for delivering the pharmaceutical formulations of the DNA targeting system for providing genetic constructs and/or proteins of the DNA targeting system. The delivery of the DNA targeting system may be the transfection or electroporation of the DNA targeting system as one or more nucleic acid molecules that is expressed in the cell and delivered to the surface of the cell. The DNA targeting system protein may be delivered to the cell. The nucleic acid molecules may be electroporated using BioRad Gene Pulser Xcell or Amaxa Nucleofector IIb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The vector encoding a DNA targeting system protein may be delivered to the mammal by DNA injection (also referred to as DNA vaccination) with and without in vivo electroporation, liposome mediated, nanoparticle facilitated, and/or recombinant vectors. The recombinant vector may be delivered by any viral mode. The viral mode may be recombinant lentivirus, recombinant adenovirus, and/or recombinant adeno-associated virus.

The nucleotide encoding a DNA targeting system protein may be introduced into a cell to induce gene expression of the target gene. For example, one or more nucleotide sequences encoding the DNA targeting system directed towards a target gene may be introduced into a mammalian cell. Upon delivery of the DNA targeting system to the cell, and thereupon the vector into the cells of the mammal, the transfected cells will express the DNA targeting system. The DNA targeting system may be administered to a mammal to induce or modulate gene expression of the target gene in a mammal. The mammal may be human, non-human primate, cow, pig, sheep, goat, antelope, bison, water buffalo, bovids, deer, hedgehogs, elephants, llama, alpaca, mice, rats, or chicken, and preferably human, cow, pig, or chicken.

### 8. ROUTES OF ADMINISTRATION

The DNA targeting system and compositions thereof may be administered to a subject by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The DNA targeting system and compositions thereof may be administered by traditional syringes, needleless injection devices, “microprojectile bombardment gone guns,” or other physical methods such as electroporation (“EP”), “hydrodynamic method”, or ultrasound. The composition may be delivered to the mammal by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus.

### 9. METHODS OF TREATING A DISEASE

The present disclosure is directed to a method of treating a subject in need thereof. The method comprises administering to the subject the composition for gene activation or repression, as described above. In some embodiments, the target cell is reprogrammed and/or differentiated using the DNA targeting system, as described above, and administered to the subject in need thereof. For example, the induction or administration of Foxp3 positive T cells may be used to reduce autoimmune disease severity, such as severity of diabetes, multiple sclerosis, asthma, inflammatory bowel disease, thyroiditis, renal disease and graft-versus-host disease.

The subject may have a disease, such as a disease selected from a variety of acute and chronic diseases including but not limited to genetic, degenerative, or autoimmune diseases and obesity related conditions. Diseases include acute and chronic immune and autoimmune pathologies, such as, but not limited to, rheumatoid arthritis (RA), juvenile chronic arthritis (JCA), tissue ischemia, thyroiditis, graft versus host disease (GVHD), scleroderma, diabetes mellitus, Graves' disease, disc degeneration and low back pain, allergy, acute or chronic immune disease associated with an allogenic transplantation, such as, but not limited to, renal transplantation, cardiac transplantation, bone marrow transplantation, liver transplantation, pancreatic transplantation, small intestine transplantation, lung transplantation and skin transplantation; infections, including, but not limited to, sepsis syndrome, cachexia, circulatory collapse and shock resulting from acute or chronic bacterial infection, acute and chronic parasitic and/or infectious diseases, bacterial, viral or fungal, such as a human immunodeficiency virus (HIV), acquired immunodeficiency syndrome (AIDS) (including symptoms of cachexia, autoimmune disorders, AIDS dementia complex and infections); inflammatory diseases, such as chronic inflammatory pathologies, including chronic inflammatory pathologies such as, but not limited to, sarcoidosis, chronic inflammatory bowel disease, ulcerative colitis, osteogenesis imperfecta, and Crohn's pathology or disease; vascular inflammatory pathologies, such as, but not limited to, disseminated intravascular coagulation, atherosclerosis, Kawasaki's pathology and vasculitis syndromes, such as, but not limited to, polyarteritis nodosa, Wegener's granulomatosis, Henoch-Schonlein purpura, giant cell arthritis and microscopic vasculitis of the kidneys; chronic active hepatitis; Sjogren's syndrome; spondyloarthropathies, such as ankylosing spondylitis, psoriatic arthritis and spondylitis, enteropathic arthritis and spondylitis, reactive arthritis and arthritis associated with inflammatory bowel disease; and uveitis; neurodegenerative diseases, including, but not limited to, demyelinating diseases, such as multiple sclerosis and acute transverse myelitis; myasthenia gravis; extrapyramidal and cerebellar disorders, such as lesions of the corticospinal system; disorders of the basal ganglia or cerebellar disorders; hyperkinetic movement disorders, such as Huntington's chorea and senile chorea; drug-induced movement disorders, such as those induced by drugs which block central nervous system (CNS) dopamine receptors; hypokinetic movement disorders, such as Parkinson's disease; progressive supranuclear palsy; cerebellar and spinocerebellar disorders, such as astructural lesions of the cerebellum; spinocerebellar degenerations (spinal ataxia, Friedreich's ataxia, cerebellar cortical degenerations, multiple systems degenerations (Mencel, Dejerine-Thomas, Shi-Drager, and MachadoJoseph)); and systemic disorders (Refsum's disease, abetalipoprotienemia, ataxia, telangiectasia, and mitochondrial multisystem disorder); disorders of the motor unit, such as neurogenic muscular atrophies (anterior horn cell degeneration, such as amyotrophic lateral sclerosis, infantile spinal muscular atrophy and juvenile spinal muscular atrophy); Alzheimer's disease; Down's syndrome in middle age; diffuse Lewy body disease; senile dementia of Lewy body type; Wernicke-Korsakoff syndrome; chronic alcoholism; primary biliary cirrhosis; cryptogenic fibrosing alveolitis and other fibrotic lung diseases; hemolytic anemia; Creutzfeldt-Jakob disease; subacute sclerosing panencephalitis, Hallervorden-Spatz disease; and dementia pugilistica, or any subset thereof; and malignant pathologies involving TNF-secreting tumors or other malignancies involving TNF, such as, but not limited to, leukemias (acute, chronic myelocytic, chronic lymphocytic and/or myelodyspastic syndrome); lymphomas (Hodgkin's and non-Hodgkin's lymphomas, such as malignant lymphomas (Burkitt's lymphoma or Mycosis fungoides)). For example, the induction or administration of Foxp3 positive or activated T cells may be used to reduce autoimmune disease severity, such as severity of diabetes, multiple sclerosis, asthma, inflammatory bowel disease, thyroiditis, renal disease and graft-versus-host disease.

### 10. TARGET CELLS

The target cell that is modulated may be a primary T-cell or cell line. T cells are a type of lymphocyte (in turn, a type of white blood cell) that play a central role in cell-mediated immunity. They can be distinguished from other lymphocytes, such as B cells and natural killer cells (NK cells), by the presence of a T-cell receptor (TCR) on the cell surface. They are called T cells because they mature in the thymus (although some also mature in the tonsils). The several subsets of T cells each have a distinct function. The majority of human T cells rearranges their alpha/beta T cell receptors, are termed alpha beta T cells, and are part of adaptive immune system. Specialized gamma delta T cells, which comprise a minority of T cells in the human body (more frequent in ruminants), have invariant TCR (with limited diversity), can effectively present antigens to other T cells and are considered to be part of the innate immune system.

The T cell includes any of a CD8-positive T cell (cytotoxic T cell: CTL), a CD4-positive T cell (helper T cell), a suppressor T cell, a regulatory T cell such as a controlling T cell, an effector cell, a naive T cell, a memory T cell, an αβT cell expressing TCR α and P chains, and a γδT cell expressing TCR γ and δ chains. The T cell includes a precursor cell of a T cell in which differentiation into a T cell is directed. Examples of “cell populations containing T cells” include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids. Further, a variety of cell populations containing T cells and derived from hematopoietic cells can be used in the present invention. These cells may have been activated by cytokine such as IL-2 in vivo or ex vivo. As these cells, any of cells collected from a living body, or cells obtained via ex vivo culture, for example, a T cell population obtained by the method of the present invention as it is, or obtained by freeze preservation, can be used.

In an embodiment, the target cell is a cell from a subject. In an embodiment, the subject is a human, e.g., a human patient. In an embodiment, the target cell is isolated from the subject. In an embodiment, the target cell is purified from a population of cells from the subject. In an embodiment, the subject has received, is receiving, or is going to receive a therapy, e.g., a therapy described herein. In an embodiment, the therapy comprises hematopoietic cell transplantation (HCT). In an embodiment, the subject has, or is at risk of having, a disorder, e.g., a disorder described herein. In an embodiment, the subject has, or is at risk of having, Graft-Versus-Host Disease (GvHD). In an embodiment, the subject has received, is receiving, or is going to receive organ transplantation. In an embodiment, the subject has, or is at risk of having, an immune disorder. In an embodiment, the subject has, or is at risk of having, a cancer. In an embodiment, the subject has, or is at risk of having, an infectious disease.

In an embodiment, the target cell is a cell from a graft. In an embodiment, the target is an immune cell from the graft. In an embodiment, the target cell is an immune cell (e.g., a T cell) that is capable mediating an immune response against a recipient of the graft. In an embodiment, the target cell is a T cell expressing an antigen binding protein or a functional fragment thereof, e.g., that is capable of binding to an immunogenic antigen expressed by a recipient of the graft. In an embodiment, the antigen binding protein is a T cell receptor (TCR). In an embodiment, the antigen binding protein is a chimeric antigen receptor.

In an embodiment, the target cell is a peripheral blood mononuclear cell (PBMC). In an embodiment, the target cell is chosen from a T cell, a B cell, a natural killer (NK) cell, a natural killer T (NKT) cell, a monocyte, a macrophage, a dendritic cell, a granulocyte, or a myeloid-derived suppressor cell (MDSC). In an embodiment, the target cell is a T cell. In an embodiment, the target cell is a PMBC-derived cell, e.g., a PMBC-derived T cell.

In an embodiment, the target cell is a stem cell. In an embodiment, the target cell is chosen from an induced pluripotent stem (iPS) cell, an embryonic stem cell, a tissue-specific stem cell (e.g., a hematopoietic stem cell), or a mesenchymal stem cell. In an embodiment, the target cell is derived from a stem cell, e.g., an iPS cell. In an embodiment, the target cell is a T cell derived from an iPS cell.

### 11. DIFFERENTIATED T CELLS

The present disclosure is directed to differentiated T cells produced using the compositions described above. The differentiated T cells are produced by contacting a target cell, as described above, with the disclosed compositions, such as the disclosed DNA targeting systems.

The target cell may be induced to differentiate into a subtype of T cell. For example, a primary or naïve T cell may be differentiated into a T_{reg}, Tₕ₁, Tₕ₁₇, or Tₕ₂ cell using the compositions and methods of the present invention. The differentiated cells may be used in intracellular pathogen and cancer defense (Tₕ₁), extracellular pathogen defense and autoimmunity (Tₕ₁₇), allergic+helminth response (Tₕ₂), Th₉ (helminth response), Th₂₂ (inflammation and bacterial defense), CD8 cytotoxic T cells, T_{fh} (follicular helper, B cell development), natural killer T cells, gamma delta T cells, and/or immune suppression (T_{reg}).

### 12. LIBRARY SCREENING OF T_{REG}-SPECIFIC DHSS

The present disclosure provides a method of screening for Treg-specific DNA hypersensitivity sites. The method includes contacting a plurality of modified target cells with a library of small guide RNAs (sgRNAs) that target a plurality of DNA hypersensitivity sites within the genome, thereby generating a plurality of test cells. The modified target cell includes the DNA targeting system, as described above.

### 13. KITS

Provided herein is a kit, which may be used to activate or repress gene expression of a target gene. The kit comprises a composition for activating or repressing gene expression, as described above, and instructions for using said composition. Instructions included in kits may be affixed to packaging material or may be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The composition for activating gene expression may include a modified AAV vector and a nucleotide sequence encoding a DNA targeting system, as described above. The DNA targeting system may include CRISPR/Cas9-based acetyltransferase, CRISPR/Cas9-based transcriptional activator, or CRISPR/Cas9-based transcriptional repressor, as described above, that specifically binds and targets a cis-regulatory region or trans-regulatory region of a target gene. The CRISPR/Cas9-based acetyltransferase, CRISPR/Cas9-based transcriptional activator, or CRISPR/Cas9-based transcriptional repressor, as described above, may be included in the kit to specifically bind and target a particular regulatory region of the target gene.

### 14. EXAMPLES

The foregoing may be better understood by reference to the following examples, which are presented for purposes of illustration and are not intended to limit the scope of the invention. The present invention has multiple aspects, illustrated by the following non-limiting examples.

### Example 1

### Design of Guide RNAs Targeting FOXP3 Regulatory Elements

DNAse I hypersensitivity tracks from the Encyclopedia of DNA elements (ENCODE) project were examined for T_{reg} and naive T cells (Tₕₒ). Regions of DNAse hypersensitivity corresponded to segments of the chromatin that are permissive to transcription factor binding and are associated with gene promoter and enhancer regions. While promoter regions are typically proximal to a gene's transcription start site, enhancer regions are found throughout the genome and are not necessarily within or adjacent to the gene on which they act.

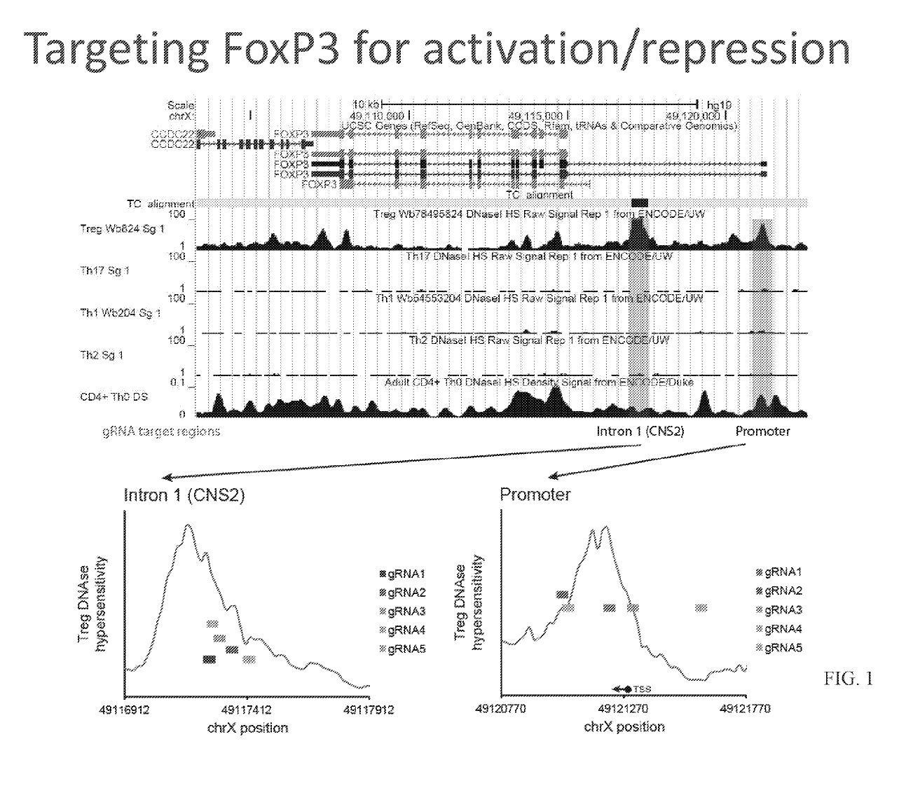

Two panels of 5 guide RNAs each were designed (Table 1): (1) a panel specific for the DNAse hypersensitive promoter region of FOXP3 (chrX:49120770-49121770), and (2) a panel specific for a DNAse hypersensitive region within intron 1 of FOXP3 (chrX:49116912-49117912), which is also referred to as the CNS2 enhancer. The DNAse I hypersensitivity of these regions is overlaid with the guide RNA binding sites in FIG. 1.

TABLE 1
FOXP3 promoter and CNS2 gRNAs

| | | | | | | |
|---|---|---|---|---|---|---|
| | | | | SEQ | | SEQ |
| | 5′ | | | ID | | ID |
| gRNA | position | Strand | Sequence | NO: | PAM | NO: |

chrX: 49120848-49121593 (promoter)

| | | | | | | |
|---|---|---|---|---|---|---|
| 1 | 49121223 | -1 | GGCTTCCACA | 1 | TGG | 11 |
| | | | CCGTACAGCG | | | |
| 2 | 49121010 | 1 | CTGGCTGGAA | 2 | TGG | 12 |
| | | | TCACGGTAGC | | | |
| 3 | 49121298 | 1 | GTGTGTGCGC | 3 | ggg | 13 |
| | | | TGATAATCAC | | | |
| 4 | 49121578 | 1 | TAAATCACAG | 4 | AGG | 14 |
| | | | GGCCAACCCG | | | |
| 5 | 49121033 | 1 | GTACATCCCA | 5 | GGG | 15 |
| | | | CTGTACCAGA | | | |

chrX: 49116764-49117884 (intron 1/CNS2)

| | | | | | | |
|---|---|---|---|---|---|---|
| 1 | 49117269 | -1 | TCGATGAAGC | 6 | CGG | 16 |
| | | | CCGGCGCATC | | | |
| 2 | 49117341 | 1 | ACAGGTTTCG | 7 | TGG | 17 |
| | | | TTCCGAGAAC | | | |
| 3 | 49117291 | 1 | GGGCTTCATC | 8 | AGG | 18 |
| | | | GACACCACGG | | | |
| 4 | 49117261 | 1 | GCCATTGACG | 9 | CGG | 19 |
| | | | TCATGGCGGC | | | |
| 5 | 49117431 | -1 | GAGCTAGGGG | 10 | TGG | 20 |
| | | | CTTGTCATAG | | | |

### Example 2

### Fluorescent Reporter Cell Line for FoxP3 Expression

Nuclease-active Cas9 from Staphylococcus pyogenes was used to add 2A-DsRed in-frame at the 3′ terminus of FOXP3 in K562 lymphoblast cells. The 2A sequence (ATNFSLLKQAGDVEENPGP (SEQ ID NO: 33)) causes ribosome skipping between the C-terminal glycine and praline residues such that the DsRed fluorophore is produced proportionally with FoxP3 rather than as a fusion protein. Puromycin was used to select for insertion of the transgene and the resulting cell line was transduced with a lentiviral vector containing S. pyogenes dCas9 fused to the core domain of the acetyltransferase p300 (SEQ ID NO: 26; Hilton et al., Nat. Biotechnol. (2015) 33:510-517). Genomic integration of both the 2A-DsRed and dCas9-p300 were confirmed by PCR.

### Example 3

### Epigenetic Control of FOXP3 Expression Using dCas9-p300

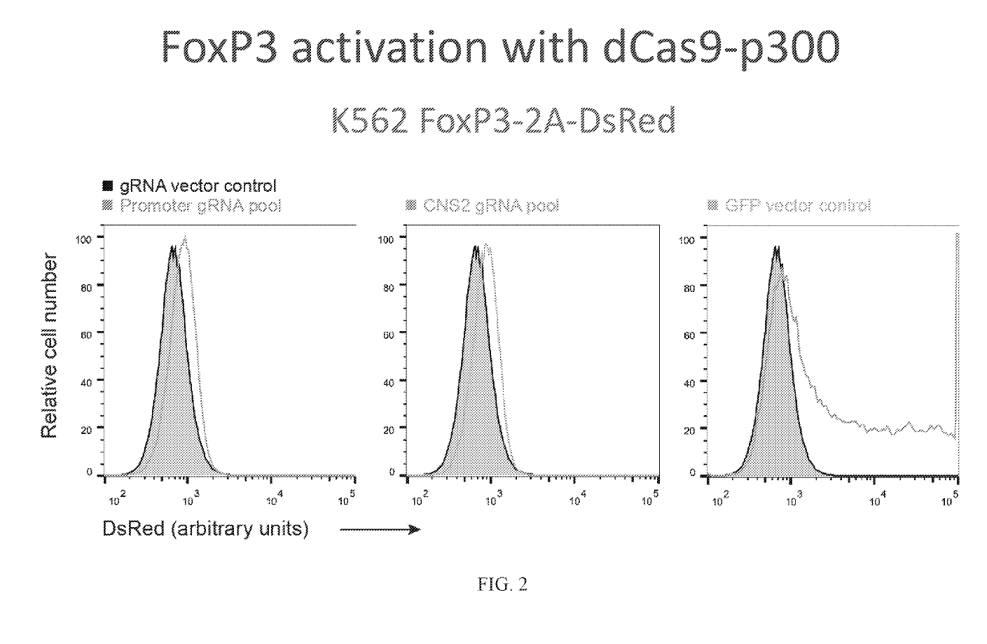

Pooled guide RNAs for either the FOXP3 promoter or the intronic DNAse hypersensitive region were transiently transfected into the K562 reporter cell line by electroporation. After 48 hours, flow cytometry was used to measure DsRed fluorescence as a marker of FoxP3 expression (FIG. 2).

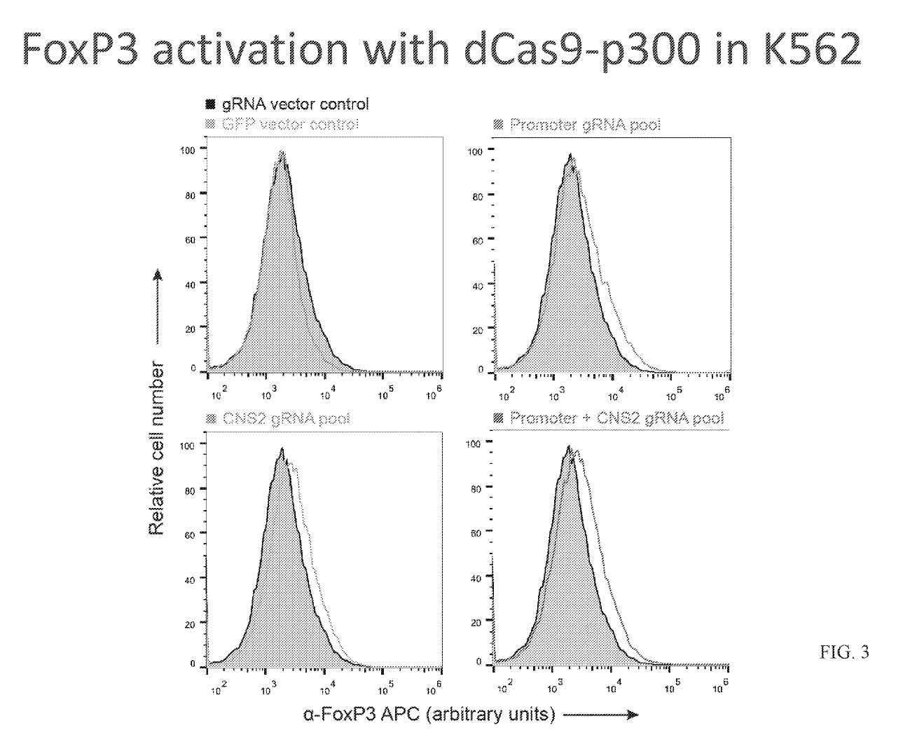

Cells that were transfected with an expression vector for a scrambled gRNA were compared to cells transfected with expression vectors for gRNAs targeting the FoxP3 promoter or intron 1 DNAse-hypersensitive sites. Cells transfected with gRNA vectors targeting both the FOXP3 promoter and the intron 1 DNAse hypersensitive site showed a measurable increase in the level of DsRed reporter fluorescence, which indicated an increase in FoxP3 expression. This was confirmed by directly staining FoxP3 with a fluorophore-tagged antibody after fixing and permeabilizing the cells (FIG. 3).

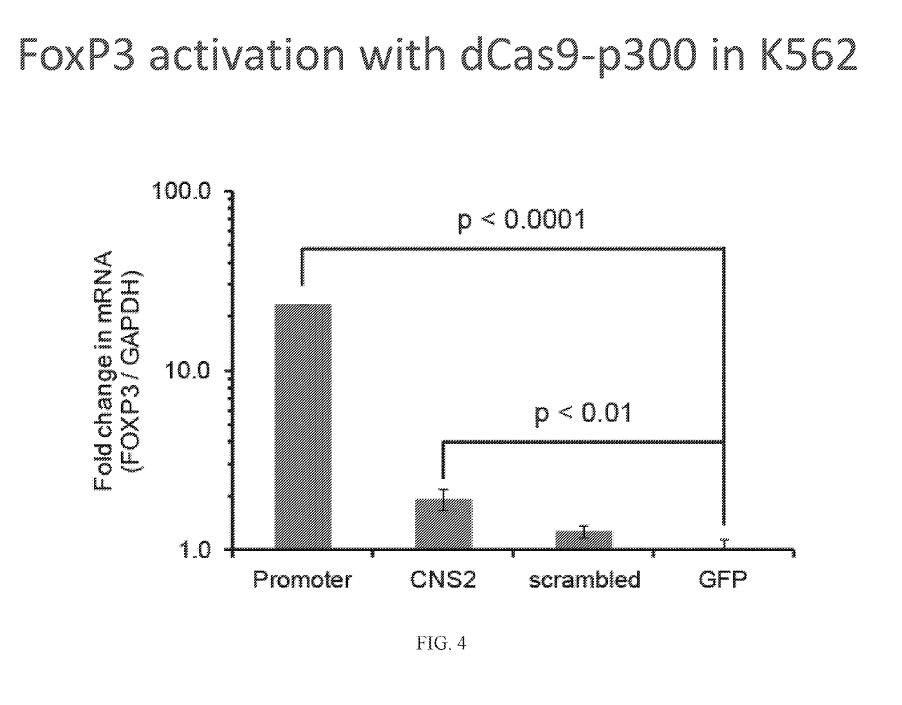

Confirming the flow cytometry results, FoxP3 mRNA was increased 23- and 1.9-fold in cells transfected with the promoter and intron 1 gRNAs, respectively, compared to cells transfected with the a GFP-expressing control vector (FIG. 4). Cells transfected with a scrambled gRNA control showed a slight increase in FoxP3 mRNA versus the group transfected with a GFP-expressing control vector that was not statistically significant (p>0.1).

### Example 4

### Library Screening of T_{reg}-Specific DHSs

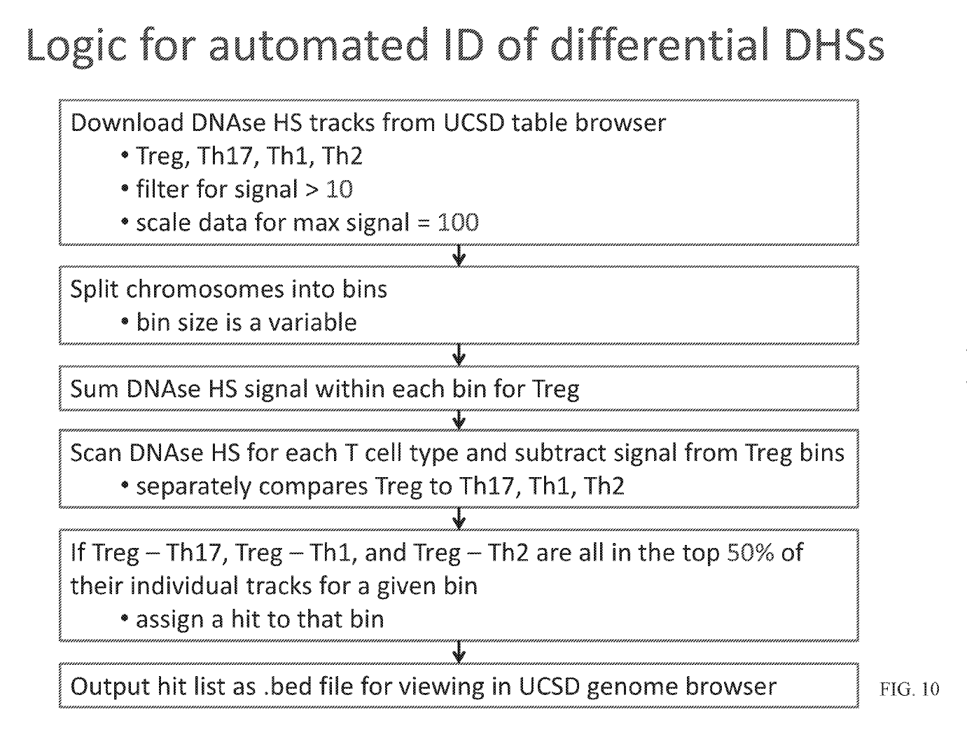

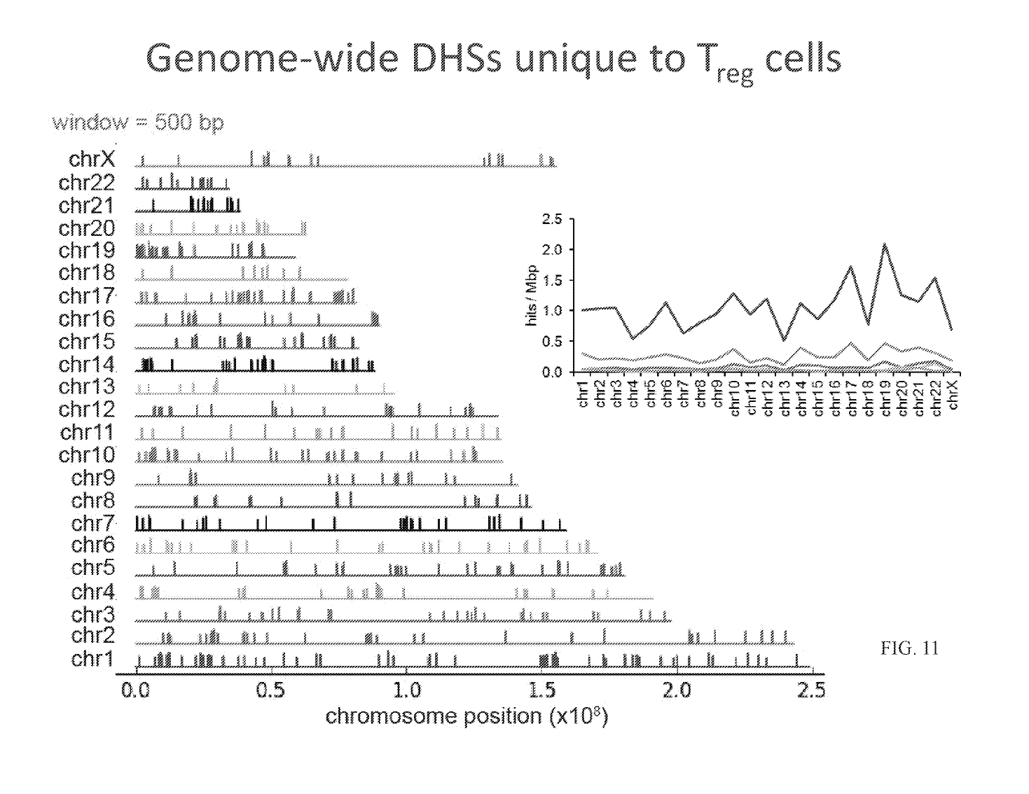

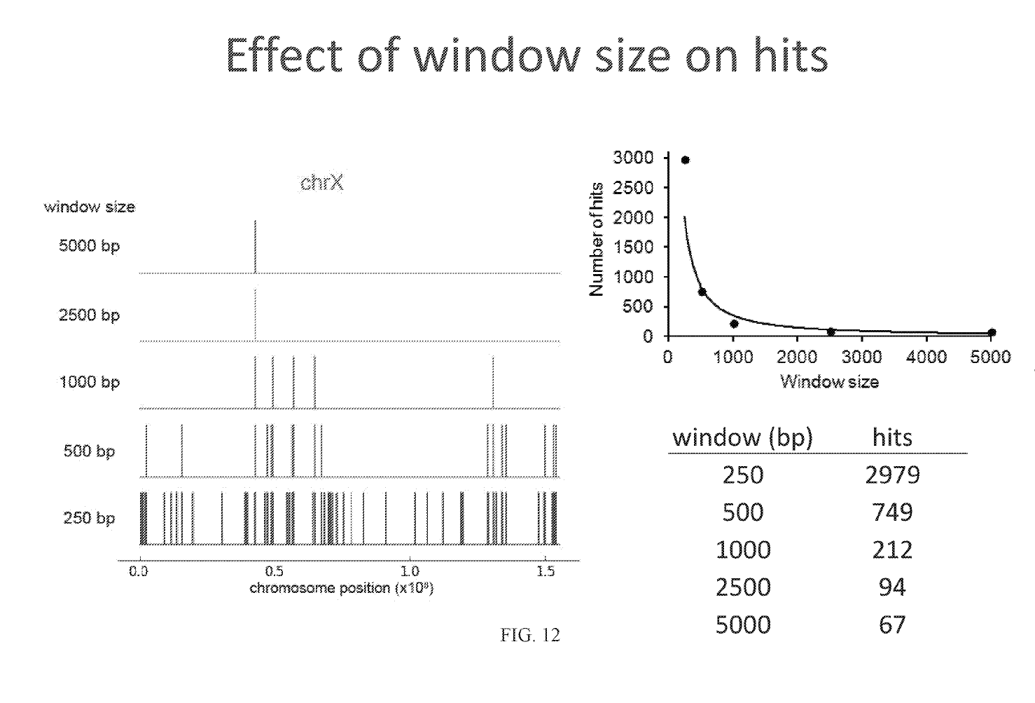

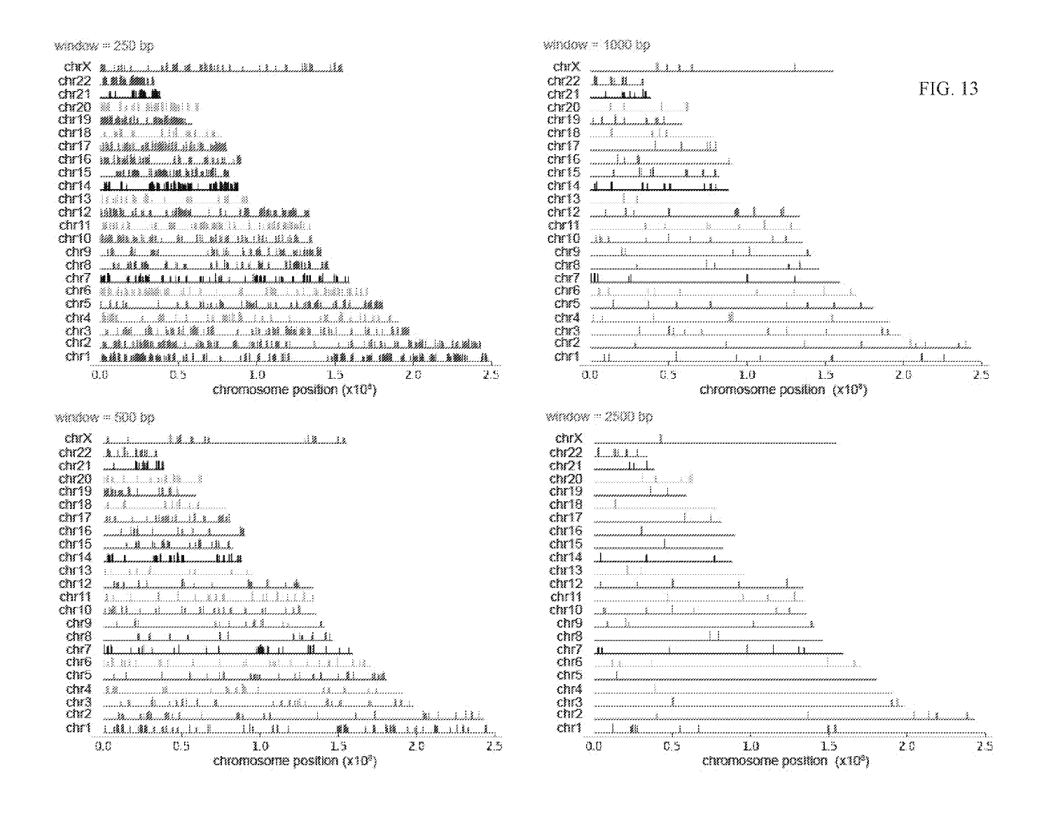

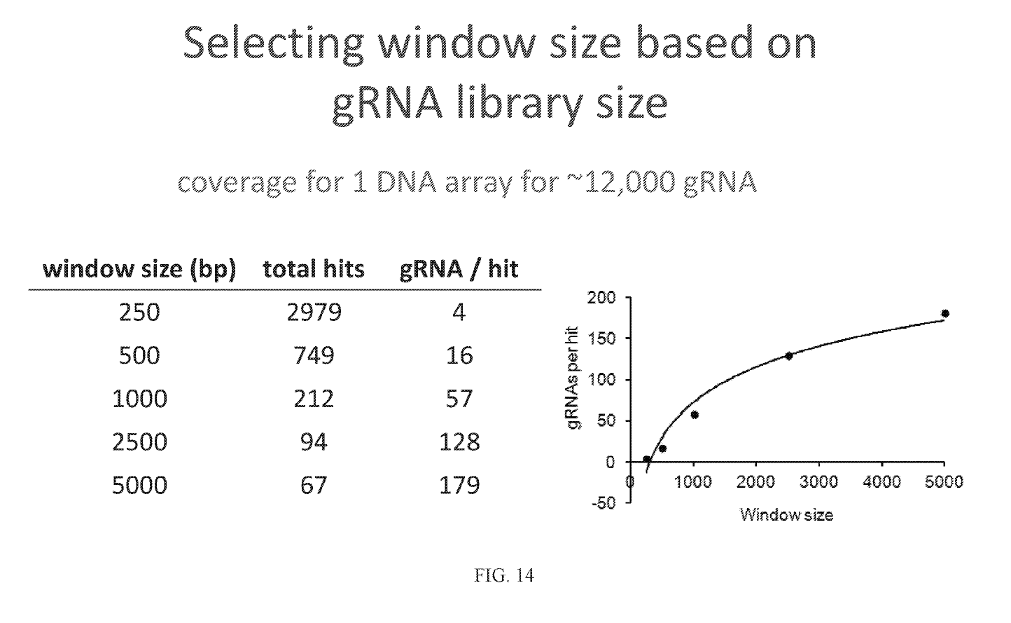

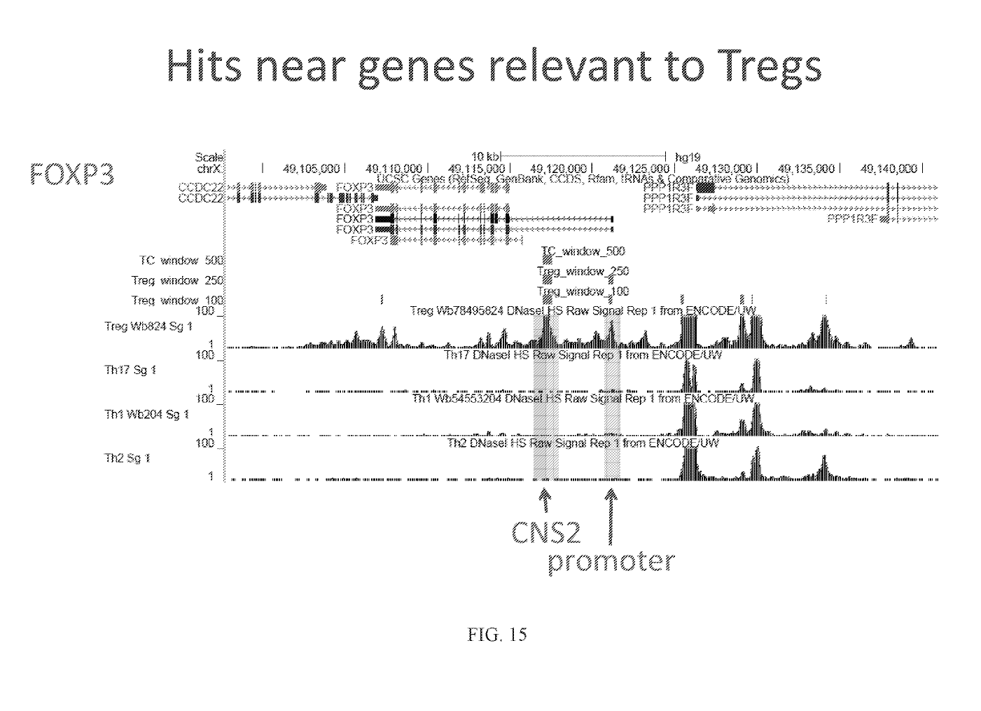

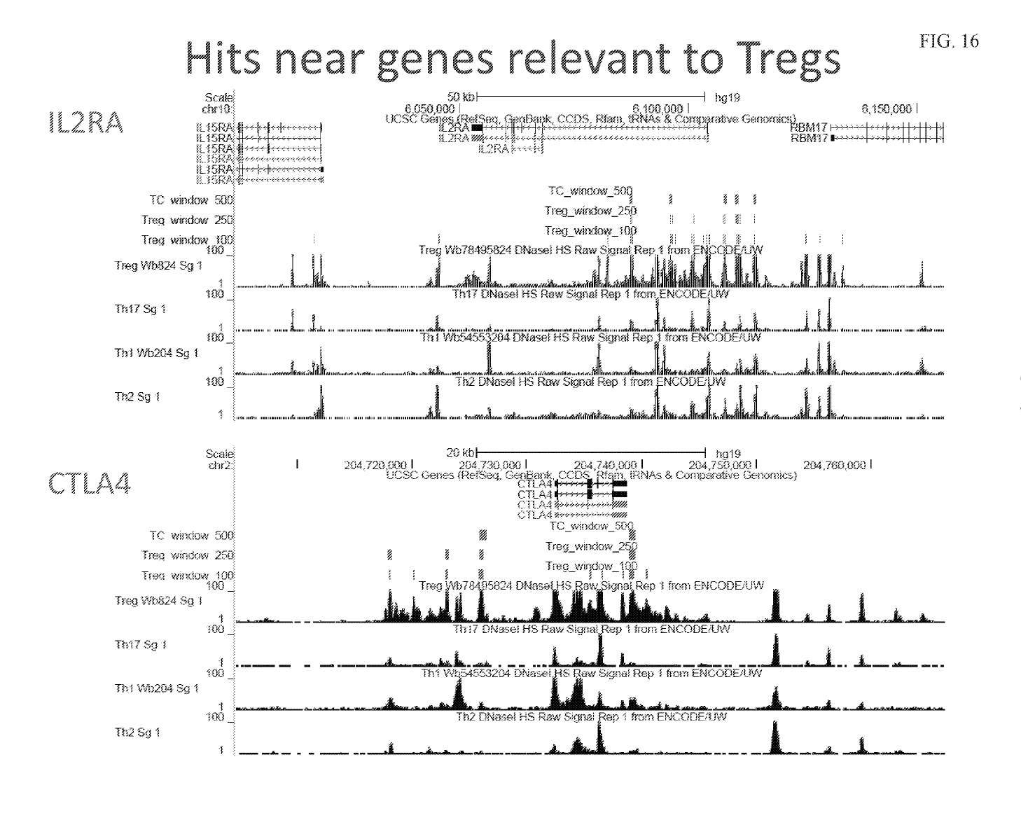

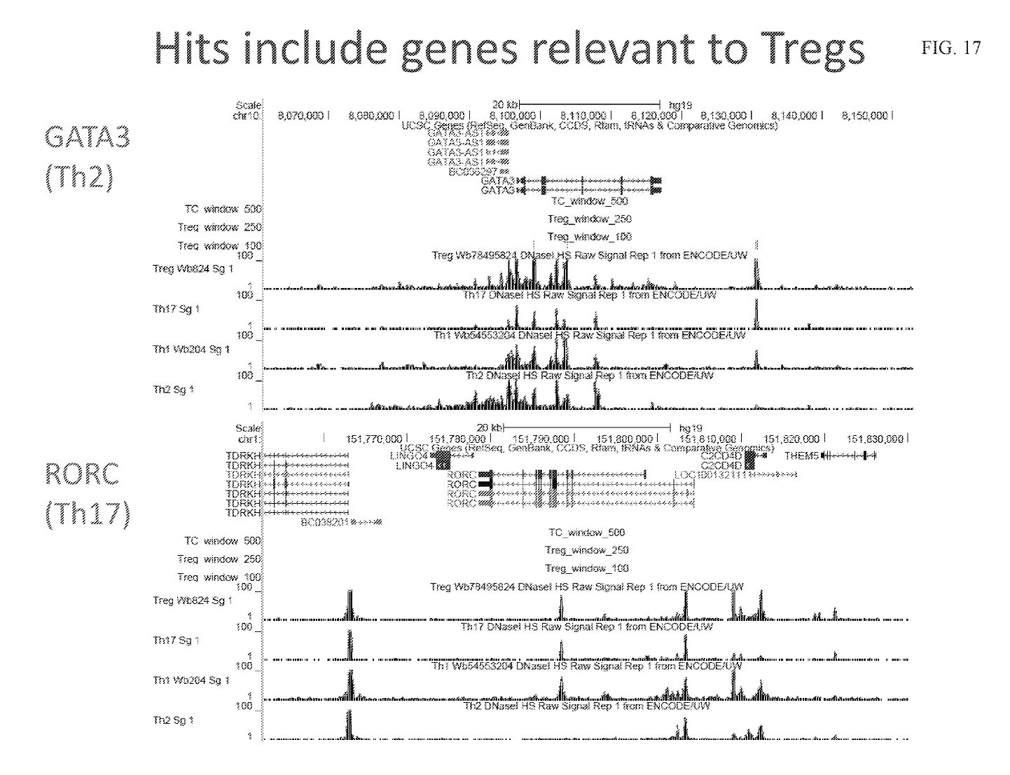

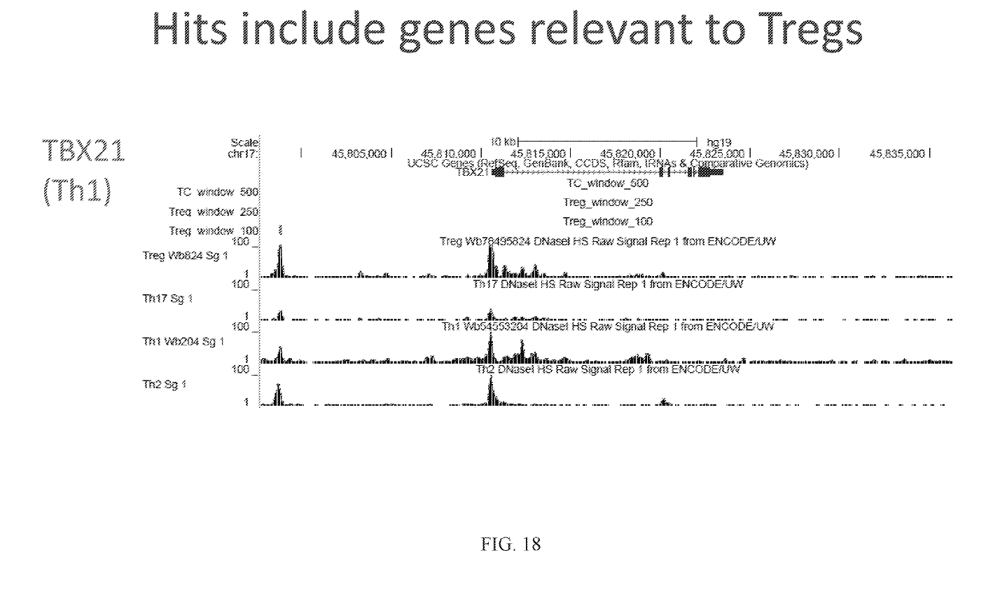

To screen genome-wide, a computational method was generated to identity unique DHSs to limit the search space. The logic for automated identification of differential DHSs is shown in FIG. 10. The algorithm in FIG. 10 was used to generate Python code that identifies “hits” that are DNAse-sensitive in Tregs, but not in Th1, Th2, or Th17 cells. These “hit” regions were screened for their ability to act as enhancers for a T cell-related gene of interest—such as FoxP3—by designing a panel of guide RNAs specific to one or more hits in parallel. The resulting guide RNA library was then introduced into T cells and the effects of individual or groups of guide RNAs on the gene of interest were assessed using one or more methods such as flow cytometry or real-time PCR. FIGS. 11-14 show the genome-wide DHSs unique to T_{reg} cells. The window size shown in FIGS. 11-14 was to widen or narrow the number of hits that was investigated. The window was the size of regions that were compared between T cell subsets. The window size to be used was affected by the number of guide RNAs that was included in a library screen, which was limited by microarray synthesis and cost. For example, if the library size was limited to 12,000 gRNAs and it was desired to have approximately 20 gRNA per “hit” site in the genome, then the window size that the program used was 500 base pairs, which returns 749 genomic regions of 500 base pairs each. FIGS. 15-17 show the DHSs that were near genes relevant to T_{reg} cells.

### Example 5

### Epigenetic Control of FOXP3 Expression in Primary Human T Cells

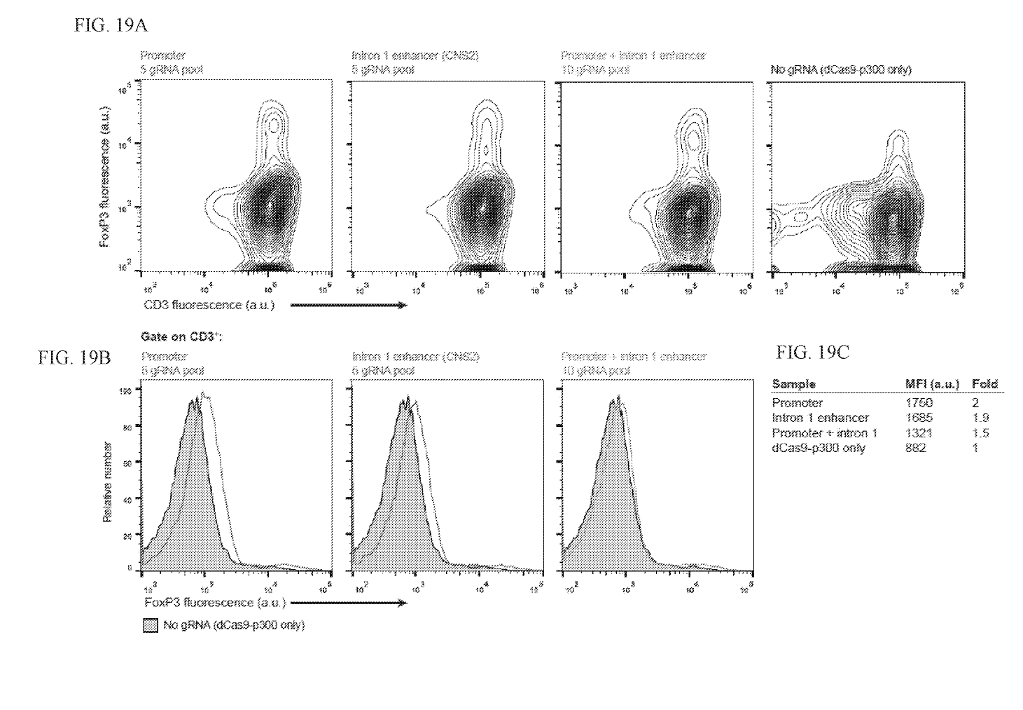

Peripheral blood mononuclear cells (PBMC) were activated overnight and co-transduced with lentiviruses encoding dCas9-p300 and pools of guide RNA expression vectors (5 guide RNAs for the FoxP3 promoter, 5 guide RNAs for the FoxP3 intron 1 enhancer, the combined 10 guide RNAs for the FoxP3 promoter and intron 1 enhancer, or no guide RNA transfer vector, see Table 1). Cells were rested 5 days after transduction then transduced cells were selected for 2 days in puromycin. At day 7 after transduction, cells were stained with anti-CD3 Alexa488 (a pan-T cell marker) and anti-FoxP3 allophycocyanin (APC). FIGS. 19A-19C show that epigenetic modification of the FOXP3 promoter or an enhancer within intron 1 induces FoxP3 expression in primary human T cells.

### Example 6

### Design of Guide RNAs Targeting CCR7 Regulatory Elements

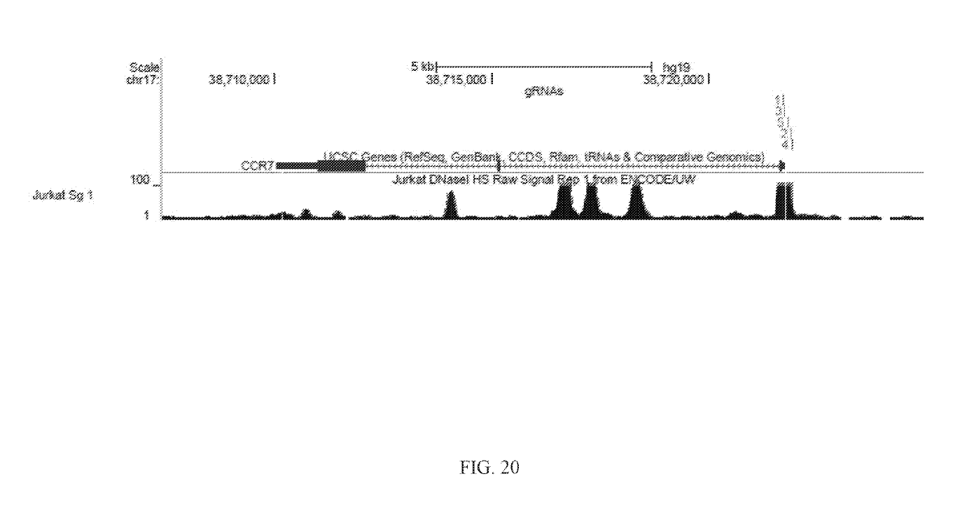

DNAse I hypersensitivity tracks from the Encyclopedia of DNA elements (ENCODE) project were examined for Jurkat cells. A panel of 5 guide RNAs was designed (Table 2). The panel was specific for the DNAse hypersensitive promoter region of CCR7. The DNAse I hypersensitivity of these regions is overlaid with the guide RNA binding sites in FIG. 20.

TABLE 2

| CCR7 gRNAs | | SEQ | | SEQ | Position | |
|---|---|---|---|---|---|---|
| | Sequence | ID | | ID | (hg19 | |
| gRNA | (5′→3′) | NO: | PAM | NO: | chr17) | Strand |
| 1 | CCCCAGACAG | 38 | AGG | 43 | 38721712 | -1 |
| | GGGTAGTGCG | | | | | |
| 2 | GGGTGACAGT | 39 | AGG | 44 | 38721895 | 1 |
| | CGCTGGTCAT | | | | | |
| 3 | GGCTTCTCCG | 40 | AGG | 45 | 38721752 | -1 |
| | ACAACTTAAA | | | | | |
| 4 | TCATAGGATC | 41 | AGG | 46 | 38721911 | 1 |
| | CTGAATCATT | | | | | |
| 5 | AGCCCTCCCT | 42 | GGG | 47 | 38721825 | 1 |
| | GACTCATGCA | | | | | |

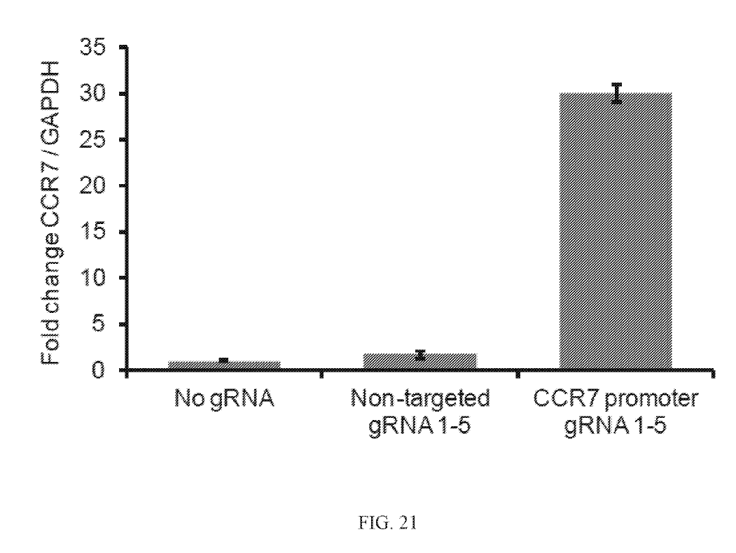

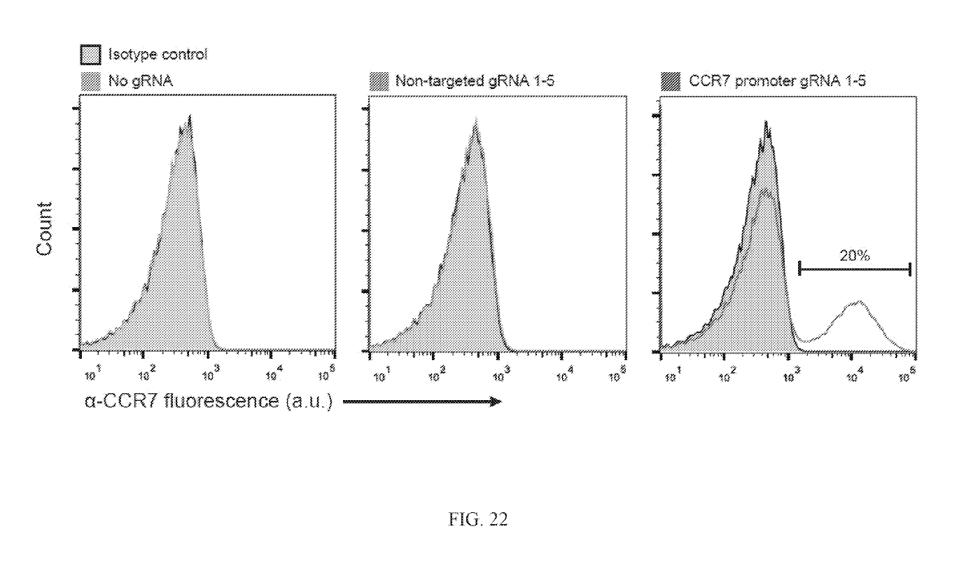

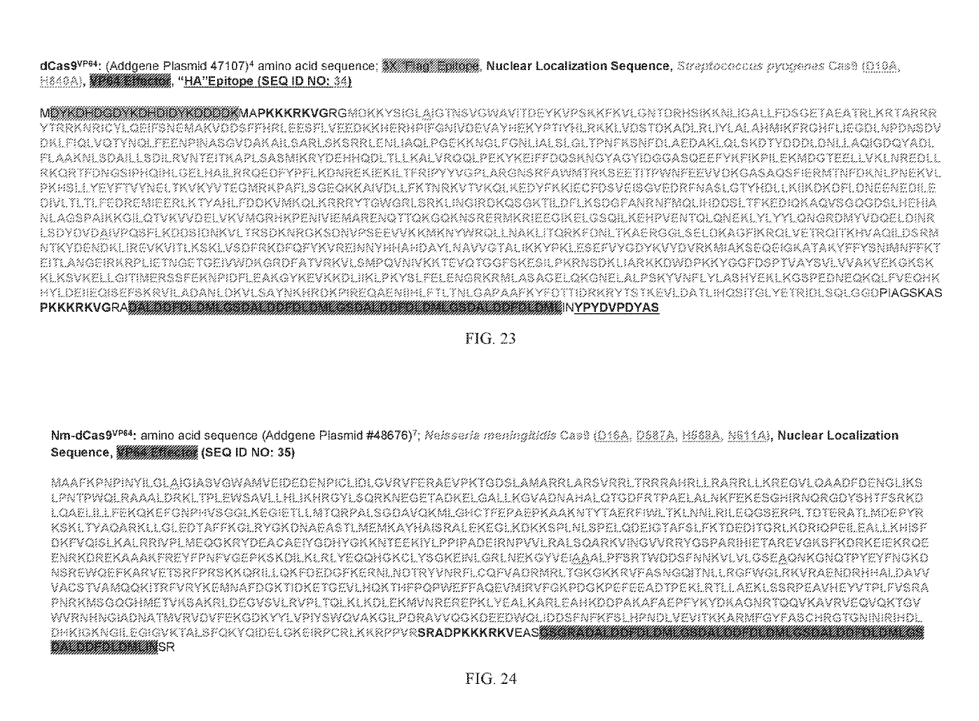

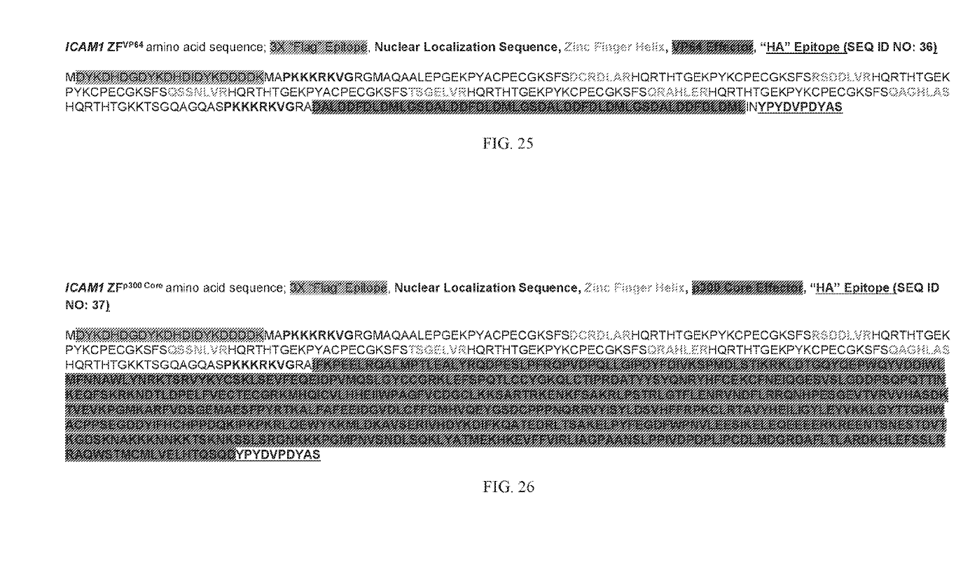

Cells that were transfected with an expression vector for a non-targeted gRNAs were compared to cells transfected with expression vectors for gRNAs targeting the CCR7 promoter DNAse-hypersensitive sites. CCR7 mRNA was increased in cells transfected with the promoter compared to cells transfected with the no gRNA or non-targeted gRNA 1-5 control (FIG. 21). Cells transfected with gRNA vectors targeting the CCR7 promoter DNAse hypersensitive site showed a measurable increase in the level of fluorescence, which indicated an increase in CCR7 expression. This was confirmed by directly staining CCR7 with a fluorophore-tagged antibody after fixing and permeabilizing the cells (FIG. 22).

### Example 7

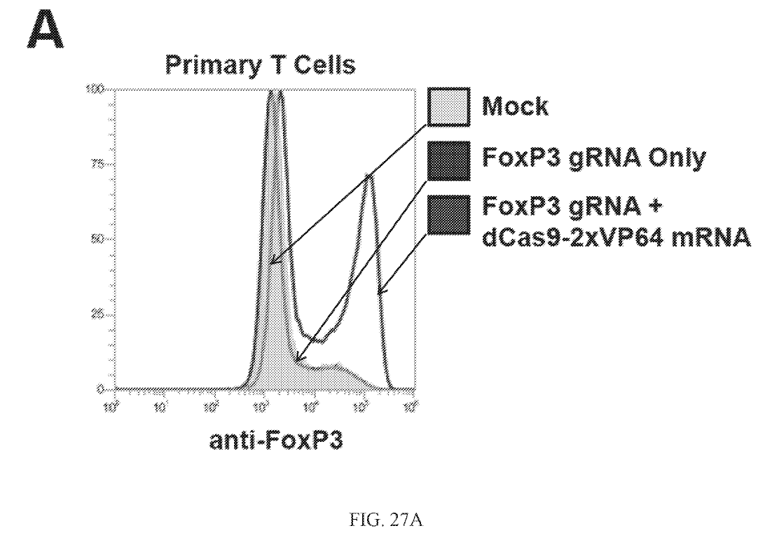

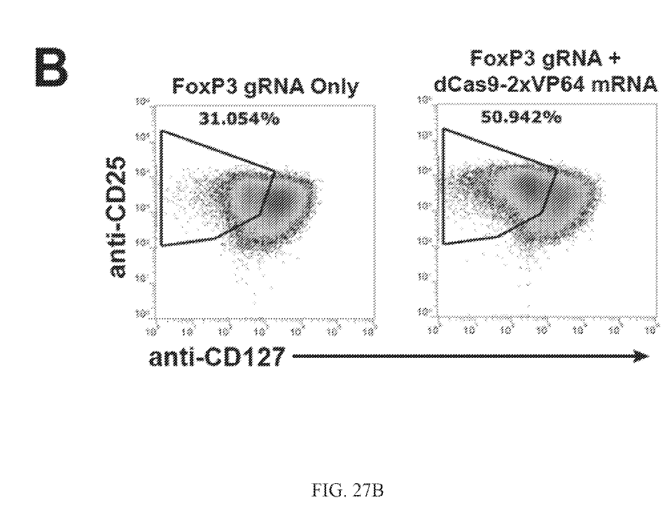

Primary T cells were isolated from buffy coats via Ficoll-paque density separation, followed by magnetic separation. The T cells were mock electroporated (gray), electroporated with synthetic FoxP3 gRNA only (red), or electroporated with synthetic FoxP3 gRNA and in vitro transcribed dCas9-2xVP64 mRNA (SEQ ID NO: 34) (blue). The synthetic FoxP3 gRNA of SEQ ID NO: 3, which targets the FoxP3 promoter, was used. Cells were fixed and stained for FoxP3 48 hrs post-electroporation (FIG. 27A). Flow cytometry of T_{reg} specific surface markers CD25 and CD127 was performed (FIG. 27B). FIG. 27B shows that FoxP3-activated primary T cells were reprogrammed to have a T_{reg} surface profile of CD25^{hi}, CD127^{lo}.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

### REFERENCES

Zheng Y, Rudensky A Y. Foxp3 in control of the regulatory T cell lineage. Nat. Immunol. 2007, 8, 457-462.
Fontenot J D, Rasmussen J P, Williams L M, Dooley J L, Farr A G, Rudensky A Y. Regulatory T cell lineage specification by the forkhead transcription factor foxp3. Immunity, 2005, 22, 329-341.
Zheng Y, Josefowicz S, Chaudhry A, Peng X P, Forbush K, Rudensky A Y. Role of conserved non-coding DNA elements in the Foxp3 gene in regulatory T-cell fate. Nature, 2010, 463, 808-812.
Tone Y, Furuuchi K, Kojima Y, Tykocinski M L, Greene M I, Tone M. Smad3 and NFAT cooperate to induce Foxp3 expression through its enhancer. Nat. Immunol., 2008, 9, 194-202.
Hilton I B, D'lppolito A M, Vockley C M, Thakore P1, Crawford G E, Reddy T E, Gersbach C A Epigenome editing by a CRISPR-Cas9-based acetyltransferase activates genes from promoters and enhancers. Nat. Biotechnol. 2015, 33, 510-517.
Crawford G E, Holt I E, Whittle J, Webb B O, Tai D, Davis S, Margulies E H, Chen Y, Bernat J A, Ginsburg D, Zhou D, Luo S, Vasicek T J, Daly M J, Wolfsberg T G, Collins F S. Genome-wide mapping of DNase hypersensitive sites using massively parallel signature sequencing (MPSS). Genome Res. 2006, 16, 123-131.
Spitz F, Furlong E E. Transcription factors: from enhancer binding to developmental control. Nat. Rev. Genet. 2012, 13, 613-626.

For reasons of completeness, various aspects of the invention are set out in the following numbered clause:

Clause 1. A DNA targeting system for programming immune cell function, the DNA targeting system comprising a fusion protein and at least one guide RNA (gRNA), the fusion protein comprising two heterologous polypeptide domains, wherein the first polypeptide domain comprises a Clustered Regularly Interspaced Short Palindromic Repeats associated (Cas) protein and the second polypeptide domain comprises a peptide having histone acetyltransferase activity, a peptide having transcription activation activity, or a peptide having transcription repressor activity, wherein the at least one gRNA targets a target region in at least one gene of FoxP3, IL2RA, CTLA4, GATA3, RORC, PDCD1, TNFRSF18, CCR7, CCR4, CXCR3, or TBX21.

Clause 2. The DNA targeting system of clause 1, wherein the at least one gRNA targets a target region of the FoxP3 gene.

Clause 3. The DNA targeting system of clause 2, wherein the second polypeptide domain comprises a peptide having histone acetyltransferase activity or transcription activation activity and the fusion protein activates transcription of the FoxP3 gene.

Clause 4. The DNA targeting system of clause 2 or 3, wherein the target region comprises an enhancer, a regulatory element, a cis-regulatory region, or a trans-regulatory region of the FoxP3 gene.

Clause 5. The DNA targeting system of clause 4, wherein the target region is a distal or proximal cis-regulatory region of the target gene.

Clause 6. The DNA targeting system of clause 4, wherein the target region is a distal or proximal trans-regulatory region of the target gene.

Clause 7. The DNA targeting system of clause 4 or 5, wherein the target region is an enhancer region or a promoter region of the target gene.

Clause 8. The DNA targeting system of any one of clauses 1-7, wherein the target region comprises a DNAse hypersensitive region.

Clause 9. The DNA targeting system of any one of clauses 1-8, wherein the target region comprises a DNAse hypersensitive region in the FoxP3 promoter or in the CNS2 enhancer element of intron 1 of the FoxP3 gene.

Clause 10. The DNA targeting system of any one of clauses 1-9, wherein the at least one gRNA comprises a 12-22 base pair complementary polynucleotide sequence of the target DNA sequence followed by a protospacer-adjacent motif.

Clause 11. The DNA targeting system of any one of clauses 1-10, wherein the at least one gRNA comprises at least one nucleotide sequence of any one of SEQ ID NOs: 11-20 or 43-47.

Clause 12. The DNA targeting system of any one of clauses 1-11, wherein the DNA targeting system comprises between one and ten different gRNAs.

Clause 13. The DNA targeting system of any one of clauses 1-12, wherein the different gRNAs bind to different target regions.

Clause 14. The DNA targeting system of any one of clauses 1-13, wherein the DNA targeting system comprises one gRNA.

Clause 15. The DNA targeting system of any one of clauses 1-14, wherein the Cas protein comprises Cas9.

Clause 16. The DNA targeting system of clause 15, wherein the Cas9 comprises at least one amino acid mutation which knocks out nuclease activity of Cas9.

Clause 17. The DNA targeting system of clause 16, wherein the Cas protein comprises an amino acid sequence of SEQ ID NO: 21 or SEQ ID NO: 22.

Clause 18. The DNA targeting system of any one of clauses 1-17, wherein the second polypeptide domain comprises a histone acetyltransferase effector domain.

Clause 19. The DNA targeting system of clause 18, wherein the histone acetyltransferase effector domain is a p300 histone acetyltransferase effector domain.

Clause 20. The DNA targeting system of any one of clauses 1-19, wherein the second polypeptide domain comprises an amino acid sequence of SEQ ID NO: 23 or SEQ ID NO: 24.

Clause 21. The DNA targeting system of any one of clauses 1-20, wherein the first polypeptide domain comprises an amino acid sequence of SEQ ID NO: 21 or SEQ ID NO: 22 and the second polypeptide domain comprises an amino acid sequence of SEQ ID NO: 23 or SEQ ID NO: 24.

Clause 22. The DNA targeting system of any one of clauses 1-21, wherein the first polypeptide domain comprises an amino acid sequence of SEQ ID NO: 21 and the second polypeptide domain comprises an amino acid sequence of SEQ ID NO. 24, or the first polypeptide domain comprises an amino acid sequence of SEQ ID NO: 22 and the second polypeptide domain comprises an amino acid sequence of SEQ ID NO. 24.

Clause 23. The DNA targeting system of any one of clauses 1-17, wherein the second polypeptide domain comprises a transactivation domain.

Clause 24. The DNA targeting system of clause 23, wherein the transactivation domain is a VP64 domain.

Clause 25. The DNA targeting system of clause 23 or 24, wherein the fusion protein comprises an amino acid sequence of SEQ ID NO: 34 or SEQ ID NO: 35.

Clause 26. The DNA targeting system of any one of clauses 1-25, further comprising a linker connecting the first polypeptide domain to the second polypeptide domain.

Clause 27. The DNA targeting system of any one of clauses 1-26, wherein the fusion protein comprises an amino acid sequence of SEQ ID NO: 25, 26, or 27.

Clause 28. A method of modulating T cell differentiation and/or function of a target cell, the method comprising contacting the target cell with the DNA targeting system of any one of clauses 1-27.

Clause 29. The method of clause 28, wherein the target cell is a primary T cell.

Clause 30. The method of clause 29, wherein the primary T cell is modulated to have an immunosuppressive phenotype.

Clause 31. The method of clause 27 or 28, wherein the primary T cell is differentiated into a Treg, Th1, Th17, or Th2 cell.

Clause 32. A method of screening of Treg-specific DNA hypersensitivity sites, the method comprising contacting a plurality of modified target cells with a library of small guide RNAs (sgRNAs) that target a plurality of DNA hypersensitivity sites within the genome, thereby generating a plurality of test cells, wherein the modified target cell comprises the DNA targeting system of any one of clauses 1-27.

Clause 33. A DNA targeting system for programming immune cell function, the DNA targeting system comprising a fusion protein, the fusion protein comprising two heterologous polypeptide domains, wherein the first polypeptide domain comprises a zinc finger protein, a TAL effector, a meganuclease, or a Clustered Regularly Interspaced Short Palindromic Repeats associated (Cas) protein and the second polypeptide domain comprises a peptide having histone acetyltransferase activity, a peptide having transcription activation activity, or a peptide having transcription repressor activity, wherein the at least one gRNA targets a target region in at least one gene of FoxP3, IL2RA, CTLA4, GATA3, RORC, PDCD1, TNFRSF18, CCR7, CCR4, CXCR3, or TBX21.

Clause 34. The DNA targeting system of clause 33, wherein the fusion protein comprises an amino acid sequence of any one of SEQ ID NO: 27, SEQ ID NO: 28, SEQ ID NO: 29, SEQ ID NO: 34, SEQ ID NO: 35, SEQ ID NO: 36, or SEQ ID NO: 37.

Clause 35. The DNA targeting system of clause 34, wherein the fusion protein comprises an amino acid sequence of any one of SEQ ID NO: 27, SEQ ID NO: 28, or SEQ ID NO: 29, and further comprises at least one gRNA.

Clause 36. The DNA targeting system of clause 35, wherein the at least one gRNA comprises a nucleotide sequence of any one of SEQ ID NOs: 11-20 or 43-47.

Clause 37. The method of any one of clauses 28-31, wherein the target cell is a human T cell

Clause 38. A differentiated T cell produced by contacting a target cell with the DNA targeting system of any one of clauses 1-27.

Clause 39. The differentiated T cell of clause 38, wherein the target cell is a primary T cell.

Clause 40. The differentiated T cell of clause 39, wherein the primary T cell is modulated to have an immunosuppressive phenotype.

Clause 41. The differentiated T cell of clause 39 or 40, wherein the primary T cell is differentiated into a T_{reg}, Tₕ₁, Tₕ₁₇, or Tₕ₂ cell.

Clause 42. The differentiated T cell of any one of clauses 38-41, wherein the target cell is a human T cell.

APPENDIX
Sequences

Streptococcus pyogenes Cas 9 (with D10A, H849A)
(SEQ ID NO: 21)
MDKKYSIGLAIGTNSVGWAVITDEYKVPSKKFKVLGNTDRHSIKKNL
IGALLFDSGETAEATRLKRTARRRYTRRKNRICYLQEIFSNEMAKVD
DSFFHRLEESFLVEEDKKHERHPIFGNIVDEVAYHEKYPTIYHLRKK
LVDSTDKADLRLIYLALAHMIKFRGHFLIEGDLNPDNSDVDKLFIQL
VQTYNQLFEENPINASGVDAKAILSARLSKSRRLENLIAQLPGEKKN
GLFGNLIALSLGLTPNFKSNFDLAEDAKLQLSKDTYDDDLDNLLAQI
GDQYADLFLAAKNLSDAILLSDILRVNTEITKAPLSASMIKRYDEHH
QDLTLLKALVRQQLPEKYKEIFFDQSKNGYAGYIDGGASQEEFYKFI
KPILEKMDGTEELLVKLNREDLLRKQRTFDNGSIPHQIHLGELHAIL
RRQEDFYPFLKDNREKIEKILTFRIPYYVGPLARGNSRFAWMTRKSE
ETITPWNFEEVVDKGASAQSFIERMTNFDKNLPNEKVLPKHSLLYEY
FTVYNELTKVKYVTEGMRKPAFLSGEQKKAIVDLLFKTNRKVTVKQL
KEDYFKKIECFDSVEISGVEDRFNASLGTYHDLLKIIKDKDFLDNEE
NEDILEDIVLTLTLFEDREMIEERLKTYAHLFDDKVMKQLKRRRYTG
WGRLSRKLINGIRDKQSGKTILDFLKSDGFANRNFMQLIHDDSLTFK
EDIQKAQVSGQGDSLHEHIANLAGSPAIKKGILQTVKVVDELVKVMG
RHKPENIVIEMARENQTTQKGQKNSRERMKRIEEGIKELGSQILKEH
PVENTQLQNEKLYLYYLQNGRDMYVDQELDINRLSDYDVDAIVPQSF
LKDDSIDNKVLTRSDKNRGKSDNVPSEEVVKKMKNYWRQLLNAKLIT
QRKFDNLTKAERGGLSELDKAGFIKRQLVETRQITKHVAQILDSRMN
TKYDENDKLIREVKVITLKSKLVSDFRKDFQFYKVREINNYHHAHDA
YLNAVVGTALIKKYPKLESEFVYGDYKVYDVRKMIAKSEQEIGKATA
KYFFYSNIMNFFKTEITLANGEIRKRPLIETNGETGEIVWDKGRDFA
TVRKVLSMPQVNIVKKTEVQTGGFSKESILPKRNSDKLIARKKDWDP
KKYGGFDSPTVAYSVLVVAKVEKGKSKKLKSVKELLGITIMERSSFE
KNPIDFLEAKGYKEVKKDLIIKLPKYSLFELENGRKRMLASAGELQK
GNELALPSKYVNFLYLASHYEKLKGSPEDNEQKQLFVEQHKHYLDEI
IEQISEFSKRVILADANLDKVLSAYNKHRDKPIREQAENIIHLFTLT
NLGAPAAFKYFDTTIDRKRYTSTKEVLDATLIHQSITGLYETRIDLS
QLGGD
Neisseria meningitidis Cas9 (with D16A, D587A,
H588A, and N611A mutations)
(SEQ ID NO: 22)
MAAFKPNPINYILGLAIGIASVGWAMVEIDEDENPICLIDLGVRVFE
RAEVPKTGDSLAMARRLARSVRRLTRRRAHRLLRARRLLKREGVLQA
ADFDENGLIKSLPNTPWQLRAAALDRKLTPLEWSAVLLHLIKHRGYL
SQRKNEGETADKELGALLKGVADNAHALQTGDFRTPAELALNKFEKE
SGHIRNQRGDYSHTFSRKDLQAELILLFEKQKEFGNPHVSGGLKEGI
ETLLMTQRPALSGDAVQKMLGHCTFEPAEPKAAKNTYTAERFIWLTK
LNNLRILEQGSERPLTDTERATLMDEPYRKSKLTYAQARKLLGLEDT
AFFKGLRYGKDNAEASTLMEMKAYHAISRALEKEGLKDKKSPLNLSP
ELQDEIGTAFSLFKTDEDITGRLKDRIQPEILEALLKHISFDKFVQI
SLKALRRIVPLMEQGKRYDEACAEIYGDHYGKKNTEEKIYLPPIPAD
EIRNPVVLRALSQARKVINGVVRRYGSPARIHIETAREVGKSFKDRK
EIEKRQEENRKDREKAAAKFREYFPNFVGEPKSKDILKLRLYEQQHG
KCLYSGKEINLGRLNEKGYVEIAAALPFSRTWDDSFNNKVLVLGSEA
QNKGNQTPYEYFNGKDNSREWQEFKARVETSRFPRSKKQRILLQKFD
EDGFKERNLNDTRYVNRFLCQFVADRMRLTGKGKKRVFASNGQITNL
LRGFWGLRKVRAENDRHHALDAVVVACSTVAMQQKITRFVRYKEMNA
FDGKTIDKETGEVLHQKTHFPQPWEFFAQEVMIRVFGKPDGKPEFEE
ADTPEKLRTLLAEKLSSRPEAVHEYVTPLFVSRAPNRKMSGQGHMET
VKSAKRLDEGVSVLRVPLTQLKLKDLEKMVNREREPKLYEALKARLE
AHKDDPAKAFAEPFYKYDKAGNRTQQVKAVRVEQVQKTGVWVRNHNG
IADNATMVRVDVFEKGDKYYLVPIYSWQVAKGILPDRAVVQGKDEED
WQLIDDSFNFKFSLHPNDLVEVITKKARMFGYFASCHRGTGNINIRI
HDLDHKIGKNGILEGIGVKTALSFQKYQIDELGKEIRPCRLKKRPPV
R
Human p300 (with L553M mutation)
(SEQ ID NO: 23)
MAENVVEPGPPSAKRPKLSSPALSASASDGTDFGSLFDLEHDLPDEL
INSTELGLTNGGDINQLQTSLGMVQDAASKHKQLSELLRSGSSPNLN
MGVGGPGQVMASQAQQSSPGLGLINSMVKSPMTQAGLTSPNMGMGTS
GPNQGPTQSTGMMNSPVNQPAMGMNTGMNAGMNPGMLAAGNGQGIMP
NQVMNGSIGAGRGRQNMQYPNPGMGSAGNLLTEPLQQGSPQMGGQTG
LRGPQPLKMGMMNNPNPYGSPYTQNPGQQIGASGLGLQIQTKTVLSN
NLSPFAMDKKAVPGGGMPNMGQQPAPQVQQPGLVTPVAQGMGSGAHT
ADPEKRKLIQQQLVLLLHAHKCQRREQANGEVRQCNLPHCRTMKNVL
NHMTHCQSGKSCQVAHCASSRQIISHWKNCTRHDCPVCLPLKNAGDK
RNQQPILTGAPVGLGNPSSLGVGQQSAPNLSTVSQIDPSSIERAYAA
LGLPYQVNQMPTQPQVQAKNQQNQQPGQSPQGMRPMSNMSASPMGVN
GGVGVQTPSLLSDSMLHSAINSQNPMMSENASVPSMGPMPTAAQPST
TGIRKQWHEDITQDLRNHLVHKLVQAIFPTPDPAALKDRRMENLVAY
ARKVEGDMYESANNRAEYYHLLAEKIYKIQKELEEKRRTRLQKQNML
PNAAGMVPVSMNPGPNMGQPQPGMTSNGPLPDPSMIRGSVPNQMMPR
ITPQSGLNQFGQMSMAQPPIVPRQTPPLQHHGQLAQPGALNPPMGYG
PRMQQPSNQGQFLPQTQFPSQGMNVTNIPLAPSSGQAPVSQAQMSSS
SCPVNSPIMPPGSQGSHIHCPQLPQPALHQNSPSPVPSRTPTPHHTP
PSIGAQQPPATTIPAPVPTPPAMPPGPQSQALHPPPRQTPTPPTTQL
PQQVQPSLPAAPSADQPQQQPRSQQSTAASVPTPTAPLLPPQPATPL
SQPAVSIEGQVSNPPSTSSTEVNSQAIAEKQPSQEVKMEAKMEVDQP
EPADTQPEDISESKVEDCKMESTETEERSTELKTEIKEEEDQPSTSA
TQSSPAPGQSKKKIFKPEELRQALMPTLEALYRQDPESLPFRQPVDP
QLLGIPDYFDIVKSPMDLSTIKRKLDTGQYQEPWQYVDDIWLMFNNA
WLYNRKTSRVYKYCSKLSEVFEQEIDPVMQSLGYCCGRKLEFSPQTL
CCYGKQLCTIPRDATYYSYQNRYHFCEKCFNEIQGESVSLGDDPSQP
QTTINKEQFSKRKNDTLDPELFVECTECGRKMHQICVLHHEIIWPAG
FVCDGCLKKSARTRKENKFSAKRLPSTRLGTFLENRVNDFLRRQNHP
ESGEVTVRVVHASDKTVEVKPGMKARFVDSGEMAESFPYRTKALFAF
EEIDGVDLCFFGMHVQEYGSDCPPPNQRRVYISYLDSVHFFRPKCLR
TAVYHEILIGYLEYVKKLGYTTGHIWACPPSEGDDYIFHCHPPDQKI
PKPKRLQEWYKKMLDKAVSERIVHDYKDIFKQATEDRLTSAKELPYF
EGDFWPNVLEESIKELEQEEEERKREENTSNESTDVTKGDSKNAKKK
NNKKTSKNKSSLSRGNKKKPGMPNVSNDLSQKLYATMEKHKEVFFVI
RLIAGPAANSLPPIVDPDPLIPCDLMDGRDAFLTLARDKHLEFSSLR
RAQWSTMCMLVELHTQSQDRFVYTCNECKHHVETRWHCTVCEDYDLC
ITCYNTKNHDHKMEKLGLGLDDESNNQQAAATQSPGDSRRLSIQRCI
QSLVHACQCRNANCSLPSCQKMKRVVQHTKGCKRKTNGGCPICKQLI
ALCCYHAKHCQENKCPVPFCLNIKQKLRQQQLQHRLQQAQMLRRRMA
SMQRTGVVGQQQGLPSPTPATPTTPTGQQPTTPQTPQPTSQPQPTPP
NSMPPYLPRTQAAGPVSQGKAAGQVTPPTPPQTAQPPLPGPPPAAVE
MAMQIQRAAETQRQMAHVQIFQRPIQHQMPPMTPMAPMGMNPPPMTR
GPSGHLEPGMGPTGMQQQPPWSQGGLPQPQQLQSGMPRPAMMSVAQH
GQPLNMAPQPGLGQVGISPLKPGTVSQQALQNLLRTLRSPSSPLQQQ
QVLSILHANPQLLAAFIKQRAAKYANSNPQPIPGQPGMPQGQPGLQP
PTMPGQQGVHSNPAMQNMNPMQAGVQRAGLPQQQPQQQLQPPMGGMS
PQAQQMNMNHNTMPSQFRDILRRQQMMQQQQQQGAGPGIGPGMANRN
QFQQPQGVGYPPQQQQRMQHHMQQMQQGNMGQIGQLPQALGAEAGAS
LQAYQQRLLQQQMGSPVQPNPMSPQQHMLPNQAQSPHLQGQQIPNSL
SNQVRSPQPVPSPRPQSQPPHSSPSPRMQPQPSPHHVSPQTSSPHPG
LVAAQANPMEQGHFASPDQNSMLSQLASNPGMANLHGASATDLGLST
DNSDLNSNLSQSTLDIH
p300 Core Effector (aa 1048-1664 of
SEQ ID NO: 22)
(SEQ ID NO: 24)
IFKPEELRQALMPTLEALYRQDPESLPFRQPVDPQLLGIPDYFDIVK
SPMDLSTIKRKLDTGQYQEPWQYVDDIWLMFNNAWLYNRKTSRVYKY
CSKLSEVFEQEIDPVMQSLGYCCGRKLEFSPQTLCCYGKQLCTIPRD
ATYYSYQNRYHFCEKCFNEIQGESVSLGDDPSQPQTTINKEQFSKRK
NDTLDPELFVECTECGRKMHQICVLHHEIIWPAGFVCDGCLKKSART
RKENKFSAKRLPSTRLGTFLENRVNDFLRRQNHPESGEVTVRVVHAS
DKTVEVKPGMKARFVDSGEMAESFPYRTKALFAFEEIDGVDLCFFGM
HVQEYGSDCPPPNQRRVYISYLDSVHFFRPKCLRTAVYHEILIGYLE
YVKKLGYTTGHIWACPPSEGDDYIFHCHPPDQKIPKPKRLQEWYKKM
LDKAVSERIVHDYKDIFKQATEDRLTSAKELPYFEGDFWPNVLEESI
KELEQEEEERKREENTSNESTDVTKGDSKNAKKKNNKKTSKNKSSLS
RGNKKKPGMPNVSNDLSQKLYATMEKHKEVFFVIRLIAGPAANSLPP
IVDPDPLIPCDLMDGRDAFLTLARDKHLEFSSLRRAQWSTMCMLVEL
HTQSQD
3X “Flag” Epitope
(SEQ ID NO: 28)
DYKDHDGDYKDHDIDYKDDDDK
Nuclear Localization Sequence
(SEQ ID NO: 29)
PKKKRKVG
HA Epitope
(SEQ ID NO: 30)
YPYDVPDYAS
Streptococcus pyogenes Cas 9 (with D10A, H849A)
(SEQ ID NO: 31)
MDKKYSIGLAIGTNSVGWAVITDEYKVPSKKFKVLGNTDRHSIKKNL
IGALLFDSGETAEATRLKRTARRRYTRRKNRICYLQEIFSNEMAKVD
DSFFHRLEESFLVEEDKKHERHPIFGNIVDEVAYHEKYPTIYHLRKK
LVDSTDKADLRLIYLALAHMIKFRGHFLIEGDLNPDNSDVDKLFIQL
VQTYNQLFEENPINASGVDAKAILSARLSKSRRLENLIAQLPGEKKN
GLFGNLIALSLGLTPNFKSNFDLAEDAKLQLSKDTYDDDLDNLLAQI
GDQYADLFLAAKNLSDAILLSDILRVNTEITKAPLSASMIKRYDEHH
QDLTLLKALVRQQLPEKYKEIFFDQSKNGYAGYIDGGASQEEFYKFI
KPILEKMDGTEELLVKLNREDLLRKQRTFDNGSIPHQIHLGELHAIL
RRQEDFYPFLKDNREKIEKILTFRIPYYVGPLARGNSRFAWMTRKSE
ETITPWNFEEVVDKGASAQSFIERMTNFDKNLPNEKVLPKHSLLYEY
FTVYNELTKVKYVTEGMRKPAFLSGEQKKAIVDLLFKTNRKVTVKQL
KEDYFKKIECFDSVEISGVEDRFNASLGTYHDLLKIIKDKDFLDNEE
NEDILEDIVLTLTLFEDREMIEERLKTYAHLFDDKVMKQLKRRRYTG
WGRLSRKLINGIRDKQSGKTILDFLKSDGFANRNFMQLIHDDSLTFK
EDIQKAQVSGQGDSLHEHIANLAGSPAIKKGILQTVKVVDELVKVMG
RHKPENIVIEMARENQTTQKGQKNSRERMKRIEEGIKELGSQILKEH
PVENTQLQNEKLYLYYLQNGRDMYVDQELDINRLSDYDVDAIVPQSF
LKDDSIDNKVLTRSDKNRGKSDNVPSEEVVKKMKNYWRQLLNAKLIT
QRKFDNLTKAERGGLSELDKAGFIKRQLVETRQITKHVAQILDSRMN
TKYDENDKLIREVKVITLKSKLVSDFRKDFQFYKVREINNYHHAHDA
YLNAVVGTALIKKYPKLESEFVYGDYKVYDVRKMIAKSEQEIGKATA
KYFFYSNIMNFFKTEITLANGEIRKRPLIETNGETGEIVWDKGRDFA
TVRKVLSMPQVNIVKKTEVQTGGFSKESILPKRNSDKLIARKKDWDP
KKYGGFDSPTVAYSVLVVAKVEKGKSKKLKSVKELLGITIMERSSFE
KNPIDFLEAKGYKEVKKDLIIKLPKYSLFELENGRKRMLASAGELQK
GNELALPSKYVNFLYLASHYEKLKGSPEDNEQKQLFVEQHKHYLDEI
IEQISEFSKRVILADANLDKVLSAYNKHRDKPIREQAENIIHLFTLT
NLGAPAAFKYFDTTIDRKRYTSTKEVLDATLIHQSITGLYETRIDLS
QLGGD
Neisseria meningitidis Cas9 (with D16A, D587A,
H588A, and N611A mutations)
(SEQ ID NO: 32)
MAAFKPNPINYILGLAIGIASVGWAMVEIDEDENPICLIDLGVRVFE
RAEVPKTGDSLAMARRLARSVRRLTRRRAHRLLRARRLLKREGVLQA
ADFDENGLIKSLPNTPWQLRAAALDRKLTPLEWSAVLLHLIKHRGYL
SQRKNEGETADKELGALLKGVADNAHALQTGDFRTPAELALNKFEKE
SGHIRNQRGDYSHTFSRKDLQAELILLFEKQKEFGNPHVSGGLKEGI
ETLLMTQRPALSGDAVQKMLGHCTFEPAEPKAAKNTYTAERFIWLTK
LNNLRILEQGSERPLTDTERATLMDEPYRKSKLTYAQARKLLGLEDT
AFFKGLRYGKDNAEASTLMEMKAYHAISRALEKEGLKDKKSPLNLSP
ELQDEIGTAFSLFKTDEDITGRLKDRIQPEILEALLKHISFDKFVQI
SLKALRRIVPLMEQGKRYDEACAEIYGDHYGKKNTEEKIYLPPIPAD
EIRNPVVLRALSQARKVINGVVRRYGSPARIHIETAREVGKSFKDRK
EIEKRQEENRKDREKAAAKFREYFPNFVGEPKSKDILKLRLYEQQHG
KCLYSGKEINLGRLNEKGYVEIAAALPFSRTWDDSFNNKVLVLGSEA
QNKGNQTPYEYFNGKDNSREWQEFKARVETSRFPRSKKQRILLQKFD
EDGFKERNLNDTRYVNRFLCQFVADRMRLTGKGKKRVFASNGQITNL
LRGFWGLRKVRAENDRHHALDAVVVACSTVAMQQKITRFVRYKEMNA
FDGKTIDKETGEVLHQKTHFPQPWEFFAQEVMIRVFGKPDGKPEFEE
ADTPEKLRTLLAEKLSSRPEAVHEYVTPLFVSRAPNRKMSGQGHMET
VKSAKRLDEGVSVLRVPLTQLKLKDLEKMVNREREPKLYEALKARLE
AHKDDPAKAFAEPFYKYDKAGNRTQQVKAVRVEQVQKTGVWVRNHNG
IADNATMVRVDVFEKGDKYYLVPIYSWQVAKGILPDRAVVQGKDEED
WQLIDDSFNFKFSLHPNDLVEVITKKARMFGYFASCHRGTGNINIRI
HDLDHKIGKNGILEGIGVKTALSFQKYQIDELGKEIRPCRLKKRPPV
R

What is claimed is:

1. A DNA targeting system for programming immune cell function, the DNA targeting system comprising a fusion protein and at least one guide RNA (gRNA), the fusion protein comprising two heterologous polypeptide domains, wherein the first polypeptide domain comprises a Clustered Regularly Interspaced Short Palindromic Repeats associated (Cas) protein and the second polypeptide domain comprises a peptide having histone acetyltransferase activity, a peptide having transcription activation activity, or a peptide having transcription repressor activity, wherein the at least one gRNA targets a target region in at least one gene of FoxP3, IL2RA, CTLA4, GATA3, RORC, PDCD1, TNFRSF18, CCR7, CCR4, CXCR3, or TBX21.

2. The DNA targeting system of claim 1, wherein the at least one gRNA targets a target region of the FoxP3 gene.

3. The DNA targeting system of claim 2, wherein the second polypeptide domain comprises a peptide having histone acetyltransferase activity or transcription activation activity and the fusion protein activates transcription of the FoxP3 gene.

4. The DNA targeting system of claim 2 or 3, wherein the target region comprises an enhancer, a regulatory element, a cis-regulatory region, or a trans-regulatory region of the FoxP3 gene.

5. The DNA targeting system of claim 4, wherein the target region is a distal or proximal cis-regulatory region of the target gene.

6. The DNA targeting system of claim 4, wherein the target region is a distal or proximal trans-regulatory region of the target gene.

7. The DNA targeting system of claim 4 or 5, wherein the target region is an enhancer region or a promoter region of the target gene.

8. The DNA targeting system of any one of claims 1-7, wherein the target region comprises a DNAse hypersensitive region.

9. The DNA targeting system of any one of claims 1-8, wherein the target region comprises a DNAse hypersensitive region in the FoxP3 promoter or in the CNS2 enhancer element of intron 1 of the FoxP3 gene.

10. The DNA targeting system of any one of claims 1-9, wherein the at least one gRNA comprises a 12-22 base pair complementary polynucleotide sequence of the target DNA sequence followed by a protospacer-adjacent motif.

11. The DNA targeting system of any one of claims 1-10, wherein the at least one gRNA comprises at least one nucleotide sequence of any one of SEQ ID NOs: 11-20 or 43-47.

12. The DNA targeting system of any one of claims 1-11, wherein the DNA targeting system comprises between one and ten different gRNAs.

13. The DNA targeting system of any one of claims 1-12, wherein the different gRNAs bind to different target regions.

14. The DNA targeting system of any one of claims 1-13, wherein the DNA targeting system comprises one gRNA.

15. The DNA targeting system of any one of claims 1-14, wherein the Cas protein comprises Cas9.

16. The DNA targeting system of claim 15, wherein the Cas9 comprises at least one amino acid mutation which knocks out nuclease activity of Cas9.

17. The DNA targeting system of claim 16, wherein the Cas protein comprises an amino acid sequence of SEQ ID NO: 21 or SEQ ID NO: 22.

18. The DNA targeting system of any one of claims 1-17, wherein the second polypeptide domain comprises a histone acetyltransferase effector domain.

19. The DNA targeting system of claim 18, wherein the histone acetyltransferase effector domain is a p300 histone acetyltransferase effector domain.

20. The DNA targeting system of any one of claims 1-19, wherein the second polypeptide domain comprises an amino acid sequence of SEQ ID NO: 23 or SEQ ID NO: 24.

21. The DNA targeting system of any one of claims 1-20, wherein the first polypeptide domain comprises an amino acid sequence of SEQ ID NO: 21 or SEQ ID NO: 22 and the second polypeptide domain comprises an amino acid sequence of SEQ ID NO: 23 or SEQ ID NO: 24.

22. The DNA targeting system of any one of claims 1-21, wherein the first polypeptide domain comprises an amino acid sequence of SEQ ID NO: 21 and the second polypeptide domain comprises an amino acid sequence of SEQ ID NO. 24, or the first polypeptide domain comprises an amino acid sequence of SEQ ID NO: 22 and the second polypeptide domain comprises an amino acid sequence of SEQ ID NO. 24.

23. The DNA targeting system of any one of claims 1-17, wherein the second polypeptide domain comprises a transactivation domain.

24. The DNA targeting system of claim 23, wherein the transactivation domain is a VP64 domain.

25. The DNA targeting system of claim 23 or 24, wherein the fusion protein comprises an amino acid sequence of SEQ ID NO: 34 or SEQ ID NO: 35.

26. The DNA targeting system of any one of claims 1-25, further comprising a linker connecting the first polypeptide domain to the second polypeptide domain.

27. The DNA targeting system of any one of claims 1-26, wherein the fusion protein comprises an amino acid sequence of SEQ ID NO: 25, 26, or 27.

28. A method of modulating T cell differentiation and/or function of a target cell, the method comprising contacting the target cell with the DNA targeting system of any one of claims 1-27.

29. The method of claim 28, wherein the target cell is a primary T cell.

30. The method of claim 29, wherein the primary T cell is modulated to have an immunosuppressive phenotype.

31. The method of claim 29 or 30, wherein the primary T cell is differentiated into a T_{reg}, Tₕ₁, Tₕ₁₇, or Tₕ₂ cell.

32. A method of screening of T_{reg}-specific DNA hypersensitivity sites, the method comprising contacting a plurality of modified target cells with a library of small guide RNAs (sgRNAs) that target a plurality of DNA hypersensitivity sites within the genome, thereby generating a plurality of test cells, wherein the modified target cell comprises the DNA targeting system of any one of claims 1-27.

33. A DNA targeting system for programming immune cell function, the DNA targeting system comprising a fusion protein, the fusion protein comprising two heterologous polypeptide domains, wherein the first polypeptide domain comprises a zinc finger protein, a TAL effector, a meganuclease, or a Clustered Regularly Interspaced Short Palindromic Repeats associated (Cas) protein and the second polypeptide domain comprises a peptide having histone acetyltransferase activity, a peptide having transcription activation activity, or a peptide having transcription repressor activity, wherein the at least one gRNA targets a target region in at least one gene of FoxP3, IL2RA, CTLA4, GATA3, RORC, PDCD1, TNFRSF18, CCR7, CCR4, CXCR3, or TBX21.

34. The DNA targeting system of claim 33, wherein the fusion protein comprises an amino acid sequence of any one of SEQ ID NO: 27, SEQ ID NO: 28, SEQ ID NO: 29, SEQ ID NO: 34, SEQ ID NO: 35, SEQ ID NO: 36, or SEQ ID NO: 37.

35. The DNA targeting system of claim 34, wherein the fusion protein comprises an amino acid sequence of any one of SEQ ID NO: 27, SEQ ID NO: 28, or SEQ ID NO: 29, and further comprises at least one gRNA.

36. The DNA targeting system of claim 35, wherein the at least one gRNA comprises a nucleotide sequence of any one of SEQ ID NOs: 11-20 or 43-47.

37. The method of any one of claims 28-31, wherein the target cell is a human T cell.

38. A differentiated T cell produced by contacting a target cell with the DNA targeting system of any one of claims 1-27.

39. The differentiated T cell of claim 38, wherein the target cell is a primary T cell.

40. The differentiated T cell of claim 39, wherein the primary T cell is modulated to have an immunosuppressive phenotype.

41. The differentiated T cell of claim 39 or 40, wherein the primary T cell is differentiated into a T_{reg}, Tₕ₁, Tₕ₁₇, or Tₕ₂ cell.

42. The differentiated T cell of any one of claims 38-41, wherein the target cell is a human T cell.
